(19) United States
(12) Patent Application Publication
(10) Pub. No.: US 2012/0195488 A1
(43) Pub. Date: Aug. 2, 2012

(54) Methods and Compositions Related to Cellular Assays

(75) Inventors: Brian Thomas Bennett, Heber City, UT (US); Joerg Bewersdorf, Branford, CT (US)

(73) Assignee: University of utah Research Foundation, Salt Lake City, UT (US)

(21) Appl. No.: 13/375,075

(22) PCT Filed: May 28, 2010
(86) PCT No.: PCT/US10/36594

§ 371 Date: Apr. 18, 2012

Related U.S. Application Data
(62) Division of application No. 61/217,329, filed on May 29, 2009.

(51) Int. Cl. C12Q 1/68; G01N 33/566; G06K 9/00; C12Q 1/02

(52) U.S. Cl. 382/133; 435/29; 435/6.1; 435/7.23; 435/7.1

(57) ABSTRACT

The present invention relates generally to the fields of molecular biology and cancer. More specifically, the invention concerns methods and compositions useful for diagnosing and assessing a subject's response to therapy.

### CROSS-REFERENCE TO RELATED PATENT APPLICATIONS

This application claims priority to U.S. Provisional Application No. 61/217,329, filed on May 29, 2009, which is hereby incorporated herein by reference in its entirety.

### BACKGROUND OF THE INVENTION

Individuals exhibit a high degree of variability in response to agents such as drugs, pharmaceutical compounds, and chemicals. The development of a drug or pharmaceutical compounds can take many years and cost millions of dollars. In addition, some companies use animals (e.g., mice, rabbits, dogs, cats, pigs, and cells, either immortalized, primary or as biopsied tissue etc.) to test the efficacy and toxicity of drugs and/or pharmaceutical compounds to obtain data for Phase I trials.

In humans, efficacy of a drug can be determined by observing several in vivo parameters, including but not limited to drug levels in blood, tissues, urine, and other biological fluids; enzymatic levels in tissues and organs; protein or sugar levels in blood and other biological fluids; elevation or depression in number, size, morphology, and/or function of cells (e.g., white blood cells, lymphocytes, red blood cells, etc.), tissues, or organs (e.g., liver, heart, kidney, etc.). Other physical and physiological parameters which may be useful include but are not limited to survival rate, appearance (e.g., hair loss, brightness of eyes, etc.), and behavior (e.g., eating habits, sleeping habits, etc.).

In drug discovery, valuable information can be obtained by understanding how a potential therapeutic affects a cell population. Insight may be gained exposing a compound to a stimulus (e.g., a genetic manipulation, exposure to a compound, radiation, or a field, deprivation of required substance, or other perturbation). The ability to quickly determine whether a population of cells exhibits a particular pathology or other classification provides a valuable tool in assessing the mechanism of action or toxicity of an uncharacterized stimulus that has been tested on the population of cells.

Models of various forms may be used to classify and/or predict behavior of populations of cells using a large number of previously classified cell populations. It would desirable to have additional models that are able to accurately predict or classify effects of diverse array of stimuli on the cell populations, as derived from data collected from individual cells.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### SUMMARY OF THE INVENTION

Disclosed herein is a method of evaluating an effect of an agent on a cell population, comprising: a) profiling cells in a cell population; b) contacting at least the profiled cells with an active agent; c) contacting at least the profiled cells with at least one marker agent; d) measuring the response of the active agent on the profiled cells.

Also disclosed is a method of determining an optimal dosage of an agent required to achieve a selected cellular response in a population of profiled cells, comprising: a) profiling cells in a cell population; b) contacting at least the profiled cells with a predetermined dose of an active agent; c) contacting at least the profiled cells with at least one marker agent; d) correlating marker levels with cellular viability in the profiled cells; e) comparing the correlation data of d) with data from other dosages; and g) determining the dosage required to achieve the selected cellular response in a population of profiled cells.

Also disclosed is a method of determining an optimal dosage of an agent required to achieve a selected cellular response in a population of profiled cells, comprising: a) profiling cells in a cell population; b) contacting at least the profiled cells with multiple titrations of a predetermined dose of an active agent, c) contacting at least the profiled cells with at least one marker agent; d) correlating marker levels with cellular viability, or any change in cellular behavior, (e.g. growth, morphology etc), in the profiled cells; e) comparing the correlation data of d) with data from other dosages; and g) determining the dosage required to achieve the selected cellular response in a population of profiled cells.

Also disclosed is a method of determining an optimal dosage of an agent required to achieve a selected cellular response in a population of profiled cells, comprising: a) profiling cells in a cell population; b) contacting at least the profiled cells with a predetermined dose of an active agent; c) contacting at least the profiled cells with at least one marker agent; d) correlating marker levels with cellular viability or a change in cellular behavior, (e.g. growth, morphology etc), in the profiled cells; e) comparing the correlation data of d) with data from other dosages; and g) determining the dosage required to achieve the selected cellular response in a population of profiled cells.

Also disclosed is a method for determining cellular responses in a profiled cell population, said method comprising: a) profiling cells in a cell population; b) contacting at least the profiled cells with at least one first marker agent and one second marker agent, wherein the first marker agent is a cell specific marker agent and the second marker agent is different from the first marker agent; c) contacting at least the profiled cells with an active agent; d) determining the amount of the second marker agent in the profiled cells; and e) comparing the level of the second marker agent in the profiled cells to the level of second marker agent in step b), wherein a change in the amount of second marker agent is indicative of a cellular response in the profiled cells.

Also disclosed is a method for determining cellular responses in a profiled cell population, said method comprising: a) profiling cells in a cell population; b) contacting at least the profiled cells with at least one first marker agent and one second marker agent, wherein the first marker agent is a cell specific marker agent and the second marker agent is different from the first marker agent; c) contacting at least the profiled cells with an active agent; d) determining the amount of the second marker agent in the profiled cells; and e) comparing the level of the second marker agent in the profiled cells to the level of first marker agent in step b), wherein a change in the amount of second marker agent is indicative of a cellular response in the profiled cells.

### BRIEF DESCRIPTION OF THE DRAWINGS

The accompanying drawings, which are incorporated in and constitute a part of this specification, illustrate several embodiments of the invention and together with the description, serve to explain the principles of the invention. These are non-limiting examples.

FIG. 1 shows level two of the distillation process. In FIG. 1, the following apply: A=p-ATM, H=γH2AX, S=signal, c=cells, t=time, d=drug, dd=drug dose, N=population, +=positive, < >=average.

FIG. 2 shows level three of the distillation process and the extraction of time dependence. In FIG. 2, the follow apply: A=p-ATM, H=γH2AX, S=signal, c=cells, t=time, d=drug, dd=drug dose, N=population, +=positive, < >=average.

FIG. 3 shows level four of the distillation process and the extraction of dose and drug dependence. In FIG. 3, the follow apply: A=p-ATM, H=γH2AX, S=signal, c=cells, t=time, d=drug, dd=drug dose, N=population, +=positive, < >=average.

FIG. 4 shows distillation of taxol treated MCF7 cells and the fraction of γH2AX positive cells.

FIG. 5 shows distillation of taxol treated MCF7 cells and the average p-ATM signal per cell. At 5 uM, the signal is lowest at approximately 4 hours and then there is no change. The other signals are similar and coincide with decreased cell count. All dd show varying levels of increase at 12 hours.

FIG. 6 shows distillation of taxol treated MCF7 cells and the total number of cells. All dd kill cells in a titration dependent fashion, indicating that titration should be optimized between 1 uM and 5 uM.

FIG. 7A shows level three distillation of etoposide treated HeLa cells and the pATM mean intensity. FIG. 7B shows level three distillation of etoposide treated HeLa cells and γH2AX mean intensity. FIGS. 7A-B indicate good separation in <SA>, no decrease in <SA>, no decrease in <NH+>, and no killing of cells.

FIG. 8 shows an example of the disclosed methods.

### DETAILED DESCRIPTION OF THE INVENTION

The present disclosure describes, in part, high-throughput labeled, adherent (primary, biopsied or immortalized) cell-based assays to assess the proper drug(s) or chemical, non endogenous agents needed on an individual patient basis. Currently, for example, cancer patients can be treated using a much higher dose of a combinatorial drug regime than may be necessary, compromising quality of life and rendering a weakened immune system. This can lead to complications and possible death from the treatments alone. This assay directly assesses the outcome of treatment and provides the actual level of drug needed without overdosing the patient. The disclosed assays and methods can directly assesses the outcome of treatment and provides the actual level of drug needed, to the nanomolar level, without overdosing the patient. Additionally, drugs can be looked at individually, providing information such as effectiveness or non-effectiveness of a treatment regimen for a given patient. Finally, data presented here shows that much lower use of these drugs, combined with the use of several different drugs and radiation, can be much more effective than traditional treatments. Further, computer models and devices for carrying out the methods disclosed herein are also provided.

All patents, patent applications and publications cited herein, whether supra or infra, are hereby incorporated by reference in their entireties into this application in order to more fully describe the state of the art as known to those skilled therein as of the date of the invention described and claimed herein.

It is to be understood that this invention is not limited to specific synthetic methods, or to specific recombinant biotechnology methods unless otherwise specified, or to particular reagents unless otherwise specified, to specific pharmaceutical carriers, or to particular pharmaceutical formulations or administration regimens, as such may, of course, vary. It is also to be understood that the terminology used herein is for the purpose of describing particular embodiments only and is not intended to be limiting.

### DEFINITIONS AND NOMENCLATURE

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The word “or” as used herein means any one member of a particular list and also includes any combination of members of that list.

By “sample” or “biological sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By “modulate” is meant to alter, by increase or decrease.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “treat” is meant to administer a compound or molecule of the invention to a subject, such as a human or other mammal (for example, an animal model). For example, a compound or molecule may be administered to a human who has an increased susceptibility for developing a cancer, or that has a cancer, in order to prevent or delay a worsening of the effects of the disease or condition, or to partially or fully reverse the effects of the disease.

By “prevent” is meant to minimize the chance that a subject who has an increased susceptibility for developing a disease will develop that disease.

As used herein, the term “gene” refers to polynucleotide sequences which encode protein products and encompass RNA, mRNA, cDNA, single stranded DNA, double stranded DNA and fragments thereof. Genes can include introns and exons. It is understood that the polynucleotide sequences of a gene can include complimentary sequences (e.g., cDNA).

The term “gene sequence(s)” refers to gene(s), full-length genes or any portion thereof “Gene sequences” can include natural genes or synthetic genes, or genes created through manipulation.

“Differential expression” or different expression” as used herein refers to the change in expression levels of genes, and/or proteins encoded by said genes, in cells, tissues, organs or systems upon exposure to an agent. As used herein, differential gene expression includes differential transcription and translation, as well as message stabilization. Differential gene expression encompasses both up- and down-regulation of gene expression.

“Naturally occurring” refers to an endogenous chemical moiety, such as a carbohydrate, polynucleotide or polypeptide sequence, i.e., one found in nature. Processing of naturally occurring moieties can occur in one or more steps, and these terms encompass all stages of processing including, but not limited to the metabolism of a non-active compound to an active compound. Conversely, a “non-naturally occurring” moiety refers to all other moieties, e.g., ones which do not occur in nature, such as recombinant polynucleotide sequences and non-naturally occurring carbohydrates.

As used herein, “cancerous tissue” is meant to mean a tissue that comprises neoplastic cells, exhibits an abnormal growth of cells and/or hyperproliferative cells. As used herein, the term “neoplastic” means an abnormal growth of a cell or tissue (e.g., a tumor or non-solid hyper proliferative cellular activity) which may be benign or cancerous. As used herein, “abnormal growth of cells” and/or “hyperproliferative cells” are meant to refer to cell growth independent of normal regulatory mechanisms (e.g., loss of contact inhibition), including the abnormal growth of benign and malignant cells or other neoplastic diseases. As used herein, the term “tumor” includes neoplasms that are identifiable through clinical screening or diagnostic procedures including, but not limited to, palpation, biopsy, cell proliferation index, endoscopy, mammography, digital mammography, ultrasonography, computed tomography (CT), magnetic resonance imaging (MRI), positron emission tomography (PET), radiography, radionuclide evaluation, CT- or MRI-guided aspiration cytology, and imaging-guided needle biopsy, among others. Such diagnostic techniques are well known to those skilled in the art and are described in Holland, et al., Cancer Medicine, 4th Ed., Vol. One, Williams & Wilkins, Baltimore, Md. (1997).

A “subject with cancer”, “cancer subject”, or a “subject diagnosed with cancer” is a subject with cancerous tissue.

As disclosed herein, the term “profiled cells” means cells that have been identified or characterized based on a preset, specific criteria. “Profiled cells” can be healthy or previously perturbed or manipulated cells. For example, profiled cells can be healthy cells or non-diseased cells as well as unhealthy or diseased cells depending on the needs of the assay data. (e.g. testing a drug on a healthy subject to gauge a possible negative result, alternatively one could start with diseased cells to see if recovery of a wide array of “healthy” phenotypes could be achieved, and one could try to induce from a healthy starting point a deleterious phenotype). Profiled cells may be a subpopulation of cells from a sample from a living multi-celled organism, such as a human.

“Profiling cells” means identifying or characterizing, from a population of cells, a subpopulation of cells that meet one or more specific criteria. Profiling is performed on an individual cell basis. In other words, each cell is independently “profiled” rather than a group or population of cells being identified simultaneously.

A “profiled cell population” means a group or population of cells that were individually profiled and have met the specific criteria.

The term “active agent” or “therapeutic agent” is defined as an active agent, such as drug, chemotherapeutic agent, chemical compound, etc.

“Marker agent” is an agent that is used to detect activity in the subpopulation of cells. For example, a label is a marker agent.

“At least one marker agent” means to allow for the use of multiple marker agents for multiple targets in a biochemical pathway(s). It is useful if the user knows or suspects the relevant biochemical pathway. Once the pathway is known, one or more markers attached to one or more targets in the pathway can be used to determine responsiveness.

“Measuring the response” is dictated by what types of markers are used, and how the markers are detected.

### General Techniques

The practice of the present invention will employ, unless otherwise indicated, conventional techniques of molecular biology (including recombinant techniques), microbiology, cell biology, biochemistry and immunology, immuno-fluorescence, and optical fluorescent collection methods which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press N.Y., 1994); Antibodies: A Laboratory Manual (Harlow et al., eds., 1987); Methods of Immunological Analysis (R. Masseyeff, W. H. Albert, and N. A. Staines, eds., Weinheim: VCH Verlags gesellschaft mbH, 1993); Principals and Methods in Toxicology (A. Wallace Hayes, ed., 2000); Analytical Methods in Toxicology (H. M. Stahr, 1991); and PCR Protocols in Molecular Toxicology (John P. Vanden Heuvel, ed., 1997).

### Methods

Immunofluorescence imaging has provided captivating visual evidence for numerous cellular events, from vesicular trafficking, organelle maturation and cell division to nuclear processes including the appearance of various proteins and chromatin components in distinct foci in response to DNA damaging agents. With the advent of new super-resolution microscope technologies such as 4Pi microscopy, STED Microscopy and the new wave of single molecule microscopes (PALM), standard immunofluorescence protocols deserve some reevaluation in order to take full advantage of these new technological accomplishments. Described herein are several methodological considerations that can overcome some of the limitations that can result from the use of currently applied procedures, with particular attention paid to the analysis of colocalization of fluorescent signals.

The mammalian DNA damage response is a complex signaling cascade involving the coordinated physical and functional interaction of a large body of proteins that are generally grouped into three interacting pathways: (i) DNA damage sensing, (ii) transduction and amplification of the damage sensing signal and, (iii) downstream effector activities that carry out end point functions such as DNA replication, fork stalling, chromatin remodeling and DNA repair. The complicated yet rapid and efficient action of these pathways is necessitated by a crucial overall goal—the maintenance of genome integrity in the face of continuous insult from both internal metabolic processes and exposure to external mutagens. This view is strongly supported by the fact that defects in many of these activities are associated with increased risk of cancer [M. O'Driscoll, P. A. Jeggo, Nat. Rev. Genet. 7 (2006) 45-54; J. Bartek, J. Bartkova, J. Lukas, Oncogene 26 (2007) 7773-7779; J. Bartek, J. Lukas, J. Bartkova, Cell Cycle 6 (2007) 2344-234.], and these findings have shaped the concept of the DNA damage response as a barrier to oncogenic transformation.

Multiple genetic, biochemical and now proteomic approaches have provided a vast database of information regarding the functions, post-translational modifications, e.g., phosphorylation and ubiquitination, and associations of many protein and chromatin components of these pathways in response to DNA damage [S. Matsuokaf et al, Science 316 (2007) 1160-1166; J. W. Harper, S. J. Elledge, Mol. Cell 28 (2007) 739-745; M. A. Cohn, A. D. D'Andrea, Mol. Cell 32 (2008) 306-312]. Immunofluorescence studies have played a leading role in providing significant advances to the current cellular view of the spatial and temporal dynamics of these components.

Some of the earliest studies of DNA damage repair proteins that included immunofluorescence images showed great promise for this technique in revealing visual details of the damage signaling and repair processes. The eukaryotic Dmc1 and Rad51 recombinase proteins were seen to form nuclear foci associated with chromosomes during meiosis or following exposure of cells to DNA damage [D. K. Bishop, Cell 79 (1994) 1081-1092; T. Haaf et al. Proc. Natl. Acad. Sci. USA 92 (1995) 2298-2302; M. Terasawa et al. Genes Dev. 9 (1995) 925-934], Results suggesting that these foci represented active regions of recombination. A functional ATM kinase, an immediate responder to DNA damage [Cj. Bakkenist, M. B. Kastan, Nature 421 (2003) 499-506; J. H. Lee, T. T. Paull, Science 308 (2005) 551-554; M. F. Lavin, S. Kozlov, Cell Cycle 6 (2007) 931-942], was shown to be required for formation of human Mre1 1 and Rad50 nuclear foci, suggesting a functional interplay between these proteins at the very early stages of the DNA damage response [R. S. Maser et. Al. Mol. Cell. Biol. 17 (1997)6087-6096]. Thus, the groundwork was established for further immunofluorescence efforts to analyze all features of the DNA damage response, including histone modifications, cellular and nuclear relocalization of proteins, formation of foci and various protein associations, as well as protein activation, e.g., using phospho-specific antibodies to detect activated ATM. At this point, the use of immunofluorescence methods has created an intriguing and seemingly well-choreographed set of events describing many aspects of the DNA damage response [M. F. Lavin, S. Kozlov, Cell Cycle 6 (2007) 931-942; R. S. Maser, K. J. Monsen, B. E. Nelms, J H Petrini, Mol. Cell. Biol. 17 (1997)6087-6096; C. Lukas et al., Nat. Cell Biol. 5 (2003)255-260; S. Bekker-Jensen et al. J. Cell Biol. 173 (2006) 195-206].

However, there have remained significant methodological barriers that have precluded bringing appropriate clarity to the images and molecular models derived from immunofluorescence studies. Methods relevant to the acquisition of target-specific, optically resolved immuno labeled images of DNA damage signaling and repair proteins in mammalian cells are discussed herein. Given the diversity of image acquisition tools and analyses available, combined with the unprecedented speed at which new sub-diffraction resolution microscopes are being introduced, commonly applied methods require some reexamination and retooling in order to take advantage of the improved imaging capabilities now available.

In some embodiments, the present invention involves the classification, diagnosis, prognosis of disease or prediction of outcome after administering a therapeutic agent (also referred to herein as an “active agent”) to treat a condition; exemplary conditions include cancers such as AML, MDS and MPN. In other embodiments, the invention involves monitoring and predicting the outcome of a condition after treatment with a therapeutic agent. In other embodiments, the invention involves selection of a treatment for a condition. In other embodiments, the invention involves drug screening using some of the methods described herein, to determine which drug or combination of drugs may be useful in a particular condition. Also, in some embodiments, one can test new or previously unknown chemical compounds, to assist in identifying new classes of compounds and the activity and effect on cells that can lead to the identification of new therapeutics. In other embodiments, the invention involves the identification of new druggable targets, that can be used alone or in combination with other treatments. In addition, the invention allows the selection of patients for specific target therapies. The invention allows for delineation of subpopulations of cells associated with a condition that are differentially susceptible to drugs or drug combinations. In another embodiment, the invention provides for identifying subpopulations of cells associated with a condition that have different genetic subclone origins. In another embodiment, the invention provides for the identification of a cell type, that in combination with other cell type(s), provide ratiometric or metrics that singularly or coordinately allow for surrogate identification of subpopulations of cells associated with a disease, diagnosis, prognosis, disease stage of the individual from which the cells were derived, response to treatment, monitoring and predicting outcome of disease.

Another embodiment involves the analysis of DNA damage pathways, apoptosis pathways, cell cycle pathways, drug transporter function, drug transporter expression, drug conversion into an active agent, internal cellular pH, redox potential environment, phosphorylation state of ITIM; drug activation; and signaling pathways for cytokines and growth factors. In some embodiments, the methods further comprises determining drug binding. In performing these processes, one preferred analysis method involves looking at cell signals and/or expression markers.

In some embodiments, the present invention provides methods for classification, diagnosis, prognosis of a disease, and/or prediction of outcome after administering a therapeutic agent to treat the disease by characterizing a plurality of pathways in a population of cells. In some embodiments, the present invention provides methods for classification, diagnosis, prognosis of a disease and/or prediction of outcome after administering a therapeutic agent to treat the disease by determining a drug transporter expression and/or function. In some embodiments, the present invention provides methods for classification, diagnosis, prognosis of disease and/or prediction of outcome after administering a therapeutic agent to treat the disease by determining a drug transporter expression and/or function and by characterizing one or more pathways in a population of cells. In some embodiments, the therapeutic agent is a therapeutic to treat cancer. In some embodiments, the therapeutic agent is a DNA damaging agent. In some embodiments, the therapeutic agent is an apoptosis and/or cell death inducing agent. In some embodiments, the therapeutic agent is a drug transporter substrate. In some embodiments, a treatment or a combination of treatments is chosen based on the characterization of plurality of pathways in single cells. In some embodiments, characterizing a plurality of pathways in single cells comprises determining whether apoptosis pathways, cell cycle pathways, or DNA damage pathways are functional in an individual in response to a therapeutic agent based on the activation levels of activatable elements within the pathways, where a pathway is functional if the activatable elements within the pathways change their activation state in response to the therapeutic agent. For example, when the apoptosis, cell cycle, signaling, and DNA damage pathways are functional the individual may be able to respond to treatment, and when at least one of the pathways is not functional the individual can not respond to treatment. In some embodiments, if the apoptosis and DNA damage pathways are functional the individual can respond to treatment. In some embodiments, a drug transporter expression and/or function in combination with characterization of one or more pathways is used for classification, diagnosis, prognosis of disease and prediction of outcome after administering a therapeutic agent in an individual. For example, an individual may not respond to treatment to a therapeutic agent if there is efflux of the therapeutic agent from the cell due to a drug transporter activity and due other disruptions in cellular pathways.

The characterization of pathways in conditions such as cancers may show disruptions in cellular pathways that are reflective of the inability of the cancer cells to respond to treatment. These disruptions may indicate increased proliferation, increased survival, evasion of apoptosis, insensitivity to anti-growth signals, efflux of therapeutic agents and other mechanisms, one or more of which could be the cause for the inability of the cancer cells to respond to treatment with a therapeutic agent. In some embodiments, the disruption in these pathways can be revealed by exposing a cell to one or more modulators that mimic one or more environmental cues and/or exposing a cell to a therapeutic agent.

### Therapeutic Agents

In some embodiments, the present invention provides methods and compositions for classification, diagnosis, prognosis of a condition, and/or prediction outcome after administering a therapeutic agent to treat the condition by characterizing a plurality of pathways in a population of cells. In some embodiments, one or more cells are contacted with therapeutic agent to analyze the response of one or more cells to the therapeutic agent. Responses may include primary refractory behavior (resistance), positive response (full or partial), and other indications such as intensity or duration of response. The results may be useful to determine treatment, understand whether a treatment will work, monitor treatment, modify therapeutic regimens, and to further optimize the selection of therapeutic agents which may be administered as one or a combination of agents. Hence, therapeutic regimens can be individualized and tailored according to the data obtained prior to, and at different times over the course of treatment, thereby providing a regimen that is individually appropriate. The methods of the invention provide tools useful in the treatment with a therapeutic agent of an individual afflicted with a condition, including but not limited to methods for assigning a risk group, methods of predicting an increased risk of relapse, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. In some embodiments, the therapeutic agent is a therapeutic to treat cancer. In some embodiments, the therapeutic agent is a DNA damaging agent. In some embodiments, the therapeutic agent is an apoptosis and/or cell death inducing agent. In some embodiments, the therapeutic agent is a drug transporter substrate.

In some embodiments, the invention provides methods and compositions for classification, diagnosis, prognosis of a condition, and/or prediction of outcome after administering a therapeutic agent to treat the condition by characterizing a plurality of pathways in a population of cells. In some embodiments, the invention further comprises analyzing a drug transporter expression and/or function. Therapeutic agents to treat cancer include chemotherapeutic agents, angiogenesis inhibitors; biological therapies such as interferons, interleukins, colony-stimulating factors, monoclonal antibodies, vaccines, gene therapy, and nonspecific immunomodulating agents; DNA damaging agents and apoptosis inducing agents. In some embodiments, the therapeutic agent to treat cancer is a DNA damaging agent. In some embodiments, the therapeutic agent to treat cancer is a substrate of a drug transporter.

In some embodiments, the cancer compound acts by damaging DNA through mechanisms including, but not limited to intercalation into DNA, inhibiting topoisomerase 1, inhibiting topoisomerase 2, inhibiting DNA or RNA polymerase, inhibiting DNA ligase, inhibiting ribonucleotide reductase, substituting bases, nucleotides or nucleosides or their analogues in nucleic acids, inhibiting DNA damage repair pathways, blocking the mitotic pathway, or the method in which the cancer compound acts by inducing apoptotic or necrotic cell death.

The therapeutic agent can comprise a binding element and an active component designed to induce cell death or apotosis. In some embodiments, the binding component is directed at a cell surface antigen, whereby the compound may be internalized and cleaved into the binding component and the active component. Active components can be cytotoxic agents or cancer chemotherapeutic agents. Binding agents can be antibodies, antibody fragments, such as single chain fragments, binding peptides, or any compound that can bind a specific cellular element to facilitate entry into the cell to carry the compound that acts on the cell. See Ricart, A D, and Tolcher, A W, Nat Clin Pract Oncol, 2007 April; 4(4):245-55; Singh et al., Curr Med. Chem. 2008; 15(18):1802-26.

Active compounds that can be delivered to the cell using a binding component include agents that induce cell death or apoptosis. These agents may be common cytotoxic agents that are used in cancer chemotherapy, or any other agents that are just generally toxic to cells. Example agents include targeted therapies, such as small molecules directed to biological targets.

Some compounds that contain binding elements attached to elements that can kill or render cells apoptotic are called antibody-drug conjugates. Antibodies are chosen for their ability to selectively target cells with receptors common to tumors. See DiJoseph F, Goad M E, Dougher M M, et al. Potent and specific antitumour efficacy of CMC-544, a CD22-targeted immunoconjugate of calicheamicin, against systemically disseminated B cell lymphoma. Clin Cancer Res. 2004; 10:8620-8629. Upon binding of the antibody—drug conjugate (ADC) to cells, the ADC-receptor complex is internalized into the cell, where the cytotoxic drug is released. Cytotoxic drugs are therefore selected for their potential to induce cell death from within the tumor cell. The molecules that link the antibody to the cytotoxic agent are chosen for their ability to stabilize the conjugate and thus minimize release of the drug before the ADC is internalized into the tumor cell. See Hamann P R. Monoclonal antibody drug conjugates. Expert Opin Ther Patents. 2005; 15:1087-1103; Mandler R, Kobayashi H, Hinson E R, et al. Herceptin-geldanamycin immunoconjugates: pharmacokinetics, biodistribution, and enhanced antitumor activity. Cancer Res. 2004; 64:1460-1467; and Sanderson R J, Hering M A, James S F, et al. In vivo drug-linker stability of an anti-CD30 dipeptide-linked auristatin immunoconjugate. Clin Cancer Res. 2005; 11:843-852.

In one embodiment of the method in the therapeutic agent, which may or may not be an antibody conjugated to a cytotoxic drug, including, but not limited to of Mylotarg, zarnestra, sorafenib, gefitnib, tanispomycin, trastuzumab, lepatinib. Or the compound may be selected from the group consisting of mitoxantrone, etoposide, daunorubicin, Idarubicin, idarubicin, epirubicin, Vidaza, Dacogen, Gleevec, Iressa, etoposide, AraC, staurosporine, lenalidomide, azacitadine, HydroxyUrea, decitabine, Zolinza, Rituxan, Fludarabine, Floxuridine, 5-FU, Gemcitabine, Cisplatin, ifosfamide, alkylating agents, nucleoside analogs, mechlorethamine and other nitrogen mustards, mercaptopurine, teniposide, Thioguanine, topotecan, troxacitabine, CSL-360, regrafomib, obatoclax, GDC-0152, GBL-310, ABT-263, phenoxodiol, SGI-1776, AT-101, ABT-869, NRX-5183, AC-220, AS-1411, ARRY-520, AZD-1152, AZD-4877, cediranib (Recentin), L-Vax, Sorafenib, BI-2536, BI-6727, BI-811283, cytarabine, bortezomib, alitretinoin, LOR-2040, annamycin, PR1 peptide antigen vaccine, vorinostat, MG-98, mocetinostat dihydrobromide, ubenimex, elacytarabine, midostaurin, valspodar, cyclosporin A, vatalanib (finasunate), lintuzumab, axitinib, SCH-727965, plitidepsin (Aplidine), arsenic trioxide (Trisenox), fostamatinib (tamatinib fosdium), tretinoin, sapacitabine, cladribine, clofarabine (Clolar/Clofarex/Evoltra), sunitinib (Sutent), oncohist, temozolomide (Temodar), tamibarotene, belinostat, DT388IL-3, amonafide malate (Xanafide), and Volorexin.

### Disease Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are known in the art. In some embodiments, the cellular pathway is a signaling pathway. Signaling pathways are also known in the art (see, e.g., Hunter T., Cell 100(1): 113-27 (2000); Cell Signaling Technology, Inc., 2002 Catalogue, Pathway Diagrams pgs. 232-253). A condition involving or characterized by altered physiological status may be readily identified, for example, by determining the state in a cell of one or more activatable elements, as taught herein.

In certain embodiments of the invention, the condition is a neoplastic, immunologic or hematopoietic condition. In some embodiments, the neoplastic, immunologic or hematopoietic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, sepsis, allergic diseases and disorders that include but are not limited to allergic rhinitis, allergic conjunctivitis, allergic asthma, atopic eczema, atopic dermatitis, and food allergy, immunodeficiencies including but not limited to severe combined immunodeficiency (SCID), hypereosiniphic syndrome, chronic granulomatous disease, leukocyte adhesion deficiency I and II, hyper IgE syndrome, Chediak Higashi, neutrophilias, neutropenias, aplasias, agammaglobulinemia, hyper-IgM syndromes, DiGeorge/Velocardial-facial syndromes and Interferon gamma-TH1 pathway defects, autoimmune and immune dysregulation disorders that include but are not limited to rheumatoid arthritis, diabetes, systemic lupus erythematosus, Graves' disease, Graves opthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

In some embodiments, the neoplastic or hematopoietic condition is non-B lineage derived. Examples of non-B lineage derived neoplastic or hematopoietic condition include, but are not limited to, Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B atypical immune lymphoproliferations.

In some embodiments, the neoplastic or hematopoietic condition is a B-Cell or B cell lineage derived disorder. Examples of B-Cell or B cell lineage derived neoplastic or hematopoietic condition include but are not limited to Chronic Lymphocytic Leukemia (CLL), B lymphocyte lineage leukemia, B lymphocyte lineage lymphoma, Multiple Myeloma, and plasma cell disorders, including amyloidosis and Waldenstrom's macroglobulinemia.

Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D J Immunol Methods 2003 February; 273 (1-2):3-13). The invention is not limited to diseases presently known to involve altered cellular function, but includes diseases subsequently shown to involve physiological alterations or anomalies.

In some embodiments, the present invention is directed to methods for analyzing the effects of a compound designed to treat cancer on one or more cells in a sample derived from an individual having or suspected of having a condition. Example conditions include any solid or hematological malignancy or neoplasm, for example, as well as AML, MDS, or MPN. See U.S. Ser. No. 61/085,789 for a discussion of the above diseases. Further examples include autoimmune, diabetes, cardiovascular, viral and other disease conditions. In some embodiments, the invention allows for identification of prognostically and therapeutically relevant subgroups of the conditions and prediction of the clinical course of an individual.

### Samples and Sampling

The methods involve analysis of one or more samples from an individual. An individual is any multicellular organism; in some embodiments, the individual is an animal, e.g., a mammal. In some embodiments, the individual is a human.

The sample may be any suitable type that allows for the analysis of single cells. Samples may be obtained once or multiple times from an individual. Multiple samples may be obtained from different locations in the individual (e.g., blood samples, tumor samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease, whether the result desired be positive or negative.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals measured in seconds, minutes, hours, days, months or years. For example, samples may be obtained at intervals of approximately 1, 2, 3, 4, 5, 6, 7, 8, 9, 10, 20, 30, 40, or 50 minutes; at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw or biopsy of the cancerous tissue(s)) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

One or more cells or cell types, or samples containing one or more cells or cell types, can be isolated from body samples. The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 .mu.m, as disclosed in U.S. patent application Ser. No. 09/790,673. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above. See also U.S. Pat. Nos. 7,381,535 and 7,393,656. All of the above patents and applications are incorporated by reference as stated above.

In some embodiments, the cells are cultured (for example in a simulated natural growth environment) post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%. Additionally, additives like antibiotics (e.g. penecillin and streptomycin) can be used to help maintain cell health, growth and normal division.

### Activatable and Active Elements

The methods and compositions of the invention may be employed to examine and profile the status of any activatable or constitually active element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously). The cell can be a hematopoietic cell. Examples of hematopoietic cells include but are not limited to pluripotent hematopoietic stem cells, granulocyte lineage progenitor or derived cells, monocyte lineage progenitor or derived cells, macrophage lineage progenitor or derived cells, megakaryocyte lineage progenitor or derived cells and erythroid lineage progenitor or derived cells.

As will be appreciated by those in the art, a wide variety of activation events can find use in the present invention. In general, the basic requirement is that the activation results in a change in the activatable protein that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state (e.g. de novo synthesis of a protein in response to the change)). What is important is to differentiate, using detectable events or moieties, between two or more activation states. However, in other instances an activatable element gets activated by increase expression. Thus, in those instances the increase expression of the activatable element will be measured whether or not there is a moiety between two or more activation states of the cells.

As an illustrative example, and without intending to be limited to any theory, an individual phosphorylatable site on a protein can activate or deactivate the protein. Additionally, phosphorylation of an adapter protein may promote its interaction with other components/proteins of distinct cellular signaling pathways. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. In some embodiments, the appearance, increases and decreases and subsequent disappearance of activatable elements may not coincide in time with the first activator. Thus, the activation level or levels of one or more activatable elements, optionally in conjunction with levels of one or more levels of biomolecules that may or may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes of cells that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, levels of intracellular or extracellular biomolecules, e.g., proteins, may be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, cellular redox signaling nodes are analyzed for a change in activation level. Reactive oxygen species (ROS) are involved in a variety of different cellular processes ranging from apoptosis and necrosis to cell proliferation and carcinogenesis. ROS can modify many intracellular signaling pathways including protein phosphatases, protein kinases, and transcription factors. This activity may indicate that the majority of the effects of ROS are through their actions on signaling pathways rather than via non-specific damage of macromolecules. The exact mechanisms by which redox status induces cells to proliferate or to die, and how oxidative stress can lead to processes evoking tumor formation are still under investigation. See Mates, J M et al., Arch Toxicol. 2008 May: 82(5):271-2; Galaris D., et al., Cancer Lett. 2008 Jul. 18; 266(1)21-9.

Under normal physiological conditions, a balance exists between oxidants and anti-oxidants in a redox homeostasis. Severe disturbance of this homeostasis causes the accumulation of high levels of reactive oxygen species (ROS). ROS are derived from the reduction of molecular oxygen to generate superoxide which then is converted to other ROS species. ROS are produced primarily by three sources within the cell. The first and a major site of ROS generation is the mitochondrial electron transport chain where electrons escaping from their transport complexes react with oxygen to form superoxide. A second major source of ROS production are from the NADPH oxidase (Nox) complexes, which were originally identified in phagocytes as a key component of the human innate host defense. Subsequently Nox complexes were found in a wide variety of non-phagocytic cells and tissues and contribute to signal transduction, cell proliferation and apoptosis with roles in many physiological processes. Nox consists of membrane-bound subunits that need to interact with cytoplasmic regulatory subunits including the small GTPase Rac in order to become active and produce ROS (Ushio-Fukai and Nakamura, Cancer Lett. (2008) 266 p37). There exists a family of Nox proteins and some of the family members are increased in cancer. The third source of ROS production is generated from other enzymes including xanthine oxidase, cyclooxygenases, lipoxygenases, myeloperoxidase, heme oxidase and cytochrome P450-based enzymes (Kuo., Antioxidants and Redox signaling (2009) 11 p 1). Cytokine growth factor and death receptor signaling can also lead to the production of ROS that function as second messengers playing an important role in signal transduction pathways. For example generation of peroxide transiently inhibits phosphatase activity in a variety kinase cascades (Morgan et al., Cell Research (2008) 18 p 343, Bindoli et al., Antioxidants and Redox Signaling (2008) 10 p 1549.).

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

In some embodiments, cellular pH is analyzed. See June, C H and Moore, and J S, Curr Protoc Immulon, 2004 December; Chapter 5:Unit 5.5; Leyval, D et al., Flow cytometry for the intracellular pH measurement of glutamate producing Corynebacterium glutamicum, Journal of Microbiological Methods, Volume 29, Issue 2, 1 May 1997, Pages 121-127; Weider, E D, et al., Measurement of intracellular pH using flow cytometry with carboxy-SNARF-1. Cytometry, 1993 November; 14(8):916-21; and Valli, M, et al., Intracellular pH Distribution in Saccharomyces cerevisiae Cell Populations, Analyzed by Flow Cytometry, Applied and Environmental Microbiology, March 2005, p. 1515-1521, Vol. 71, No. 3.

In some embodiments, the activatable element is the phosphorylation of immunoreceptor tyrosine-based inhibitory motif (ITIM). An immunoreceptor tyrosine-based inhibition motif (ITIM), is a conserved sequence of amino acids (S/I/V/LxYxxI/V/L) that is found in the cytoplasmic tails of many inhibitory receptors of the immune system. After ITIM-possessing inhibitory receptors interact with their ligand, their ITIM motif becomes phosphorylated by enzymes of the Src family of kinases, allowing them to recruit other enzymes such as the phosphotyrosine phosphatases SHP-1 and SHP-2, or inositol-phosphatases called SHIPs. These phosphatases can decrease or increase the activation of molecules involved in cell signaling. See Barrow A, Trowsdale J (2006). “You say ITAM and I say ITIM, let's call the whole thing off: the ambiguity of immunoreceptor signalling”. Eur J Immunol 36 (7): 1646-53. When phosphorylated, these phospho-tyrosine residues provide docking sites for the Shps which may result in transmission of inhibitory or activation signals that effect the signaling of neighboring membrane receptor complexes (Paul et al., Blood (2000 96:483).

Criteria may be used to profile a cell including, but not limited to, expression level of extracellular or intracellular markers, nuclear antigens, enzymatic activity, protein expression and localization, cell cycle analysis, chromosomal analysis, cell volume, and morphological characteristics like granularity and size of nucleus, size and redistribution of nucleoli, membrane change, the number of nuclear pores or other distinguishing characteristics. For example, myeloid cells can be further subdivided based on the expression of surface markers including but not limited to CD45, CD34, CD33, CD11B, CD14.

Alternatively, predefined classes of cells can be aggregated or grouped based upon shared characteristics that may include inclusion in one or more additional predefined class or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus, size and redistribution of nucleoli, membrane change, nuclear pore numbers or other distinguishing cellular characteristics.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a hematopoietic cell is classified according to the activation levels of a plurality of activatable elements. In some embodiments, 1, 2, 3, 4, 5, 6, 7, 8, 9, 10 or more activatable elements may be analyzed in a cell signaling pathway. In some embodiments, the activation levels of one or more activatable elements of a hematopoietic cell are correlated with a condition. In some embodiments, the activation levels of one or more activatable elements of a hematopoietic cell are correlated with a neoplastic or hematopoietic condition as described herein. Examples of hematopoietic cells include, but are not limited to, AML, MDS or MPDS cells.

In some embodiments, the activation level of one or more activatable elements in single cells in the sample is determined Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. In some embodiments, at least about 2, 3, 4, 5, 6, 7, 8, 9, 10 or more than 10 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

One example of a covalent modification is the substitution of a phosphate group for a hydroxyl group in the side chain of an amino acid (phosphorylation). A wide variety of proteins are known that recognize specific protein substrates and catalyze the phosphorylation of serine, threonine, or tyrosine residues on their protein substrates. Such proteins are generally termed “kinases.” Substrate proteins that are capable of being phosphorylated are often referred to as phosphoproteins (after phosphorylation). Once phosphorylated, a substrate phosphoprotein may have its phosphorylated residue converted back to a hydroxyl one by the action of a protein phosphatase that specifically recognizes the substrate protein. Protein phosphatases catalyze the replacement of phosphate groups by hydroxyl groups on serine, threonine, or tyrosine residues. Through the action of kinases and phosphatases a protein may be reversibly phosphorylated on a multiplicity of residues and its activity may be regulated thereby. Thus, the presence or absence of one or more phosphate groups in an activatable protein is a preferred readout in the present invention.

Another example of a covalent modification of an activatable protein is the acetylation of histones. Through the activity of various acetylases and deacetylylases the DNA binding function of histone proteins is tightly regulated. Furthermore, histone acetylation and histone deactelyation have been linked with malignant progression. See Nature, 2004 May 27; 429(6990): 457-63.

Another form of activation involves cleavage of the activatable element. For example, one form of protein regulation involves proteolytic cleavage of a peptide bond. While random or misdirected proteolytic cleavage may be detrimental to the activity of a protein, many proteins are activated by the action of proteases that recognize and cleave specific peptide bonds. Many proteins derive from precursor proteins, or pro-proteins, which give rise to a mature isoform of the protein following proteolytic cleavage of specific peptide bonds. Many growth factors are synthesized and processed in this manner, with a mature isoform of the protein typically possessing a biological activity not exhibited by the precursor form. Many enzymes are also synthesized and processed in this manner, with a mature isoform of the protein typically being enzymatically active, and the precursor form of the protein being enzymatically inactive. This type of regulation is generally not reversible. Accordingly, to inhibit the activity of a proteolytically activated protein, mechanisms other than “reattachment” must be used. Inhibitors may also be used. Among the enzymes that are proteolytically activated are serine and cysteine proteases, including cathepsins and caspases respectively.

In one embodiment, the activatable enzyme is a cysteine aspartic acid specific protease (caspase). The caspases are an important class of proteases that mediate programmed cell death (referred to in the art as “apoptosis”). Caspases are constitutively present in most cells, residing in the cytosol as a single chain proenzyme. These are activated to fully functional proteases by a first proteolytic cleavage to divide the chain into large and small caspase subunits and a second cleavage to remove the N-terminal domain. The subunits assemble into a tetramer with two active sites (Green, Cell 94:695-698, 1998). Many other proteolytically activated enzymes, known in the art as “zymogens,” also find use in the instant invention as activatable elements.

In an alternative embodiment the activation of the activatable element involves prenylation of the element. By “prenylation”, and grammatical equivalents used herein, is meant the addition of any lipid group to the element. Common examples of prenylation include the addition of farnesyl groups, geranylgeranyl groups, myristoylation and palmitoylation. In general these groups are attached via thioether linkages to the activatable element, although other attachments may be used.

In alternative embodiment, activation of the activatable element is detected as intermolecular clustering of the activatable element. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

In a preferred embodiment, the activation or signaling potential of elements is mediated by clustering, irrespective of the actual mechanism by which the element's clustering is induced. For example, elements can be activated to cluster a) as membrane bound receptors by binding to ligands (ligands including both naturally occurring or synthetic ligands), b) as membrane bound receptors by binding to other surface molecules, or c) as intracellular (non-membrane bound) receptors binding to ligands.

In a preferred embodiment the activatable elements are membrane bound receptor elements that cluster upon ligand binding such as cell surface receptors. As used herein, “cell surface receptor” refers to molecules that occur on the surface of cells, interact with the extracellular environment, and transmit or transduce (through signals) the information regarding the environment intracellularly in a manner that may modulate cellular activity directly or indirectly, e.g., via intracellular second messenger activities or transcription of specific promoters, resulting in transcription of specific genes. One class of receptor elements includes membrane bound proteins, or complexes of proteins, which are activated to cluster upon ligand binding. As is known in the art, these receptor elements can have a variety of forms, but in general they comprise at least three domains. First, these receptors have a ligand-binding domain, which can be oriented either extracellularly or intracellularly, usually the former. Second, these receptors have a membrane-binding domain (usually a transmembrane domain), which can take the form of a seven pass transmembrane domain (discussed below in connection with G-protein-coupled receptors) or a lipid modification, such as myristylation, to one of the receptor's amino acids which allows for membrane association when the lipid inserts itself into the lipid bilayer. Finally, the receptor has an signaling domain, which is responsible for propagating the downstream effects of the receptor.

In another embodiment the activatable elements cluster for signaling by contact with other surface molecules. In contrast to the receptors discussed above, these elements cluster for signaling by contact with other surface molecules, and generally use molecules presented on the surface of a second cell as ligands. Receptors of this class are important in cell-cell interactions, such mediating cell-to-cell adhesion and immunorecognition.

In one embodiment, the activatable elements are intracellular receptors capable of clustering. Elements of this class are not membrane-bound. Instead, they are free to diffuse through the intracellular matrix where they bind soluble ligands prior to clustering and signal transduction. In contrast to the previously described elements, many members of this class are capable of binding DNA after clustering to directly effect changes in RNA transcription.

In another embodiment the activatable element is a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalitic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Leukemia. See February 2004; 18(2): 356-8. SOCS1 and SHP1 hypermethylation in mantle cell lymphoma and follicular lymphoma: implications for epigenetic activation of the Jak/STAT pathway. Chim C S, Wong K Y, Loong F, Srivastava G.

In another embodiment the activatable element is a small molecule, carbohydrate, lipid or other naturally occurring or synthetic compound capable of having an activated isoform. In addition, as pointed out above, activation of these elements need not include switching from one form to another, but can be detected as the presence or absence of the compound. For example, activation of cAMP (cyclic adenosine mono-phosphate) can be detected as the presence of cAMP rather than the conversion from non-cyclic AMP to cyclic AMP.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886; 61/048,920; and Shulz et al., Current Protocols in Immunology 2007, 78:8.17.1-20.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGF.beta. receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3.alpha., GSK3.beta., Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon .beta., interferon .alpha., suppressors of cytokine signaling (SOCs), Cb1, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, .beta.-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Glycoprotein, nucleoside transporters, Ets, Elk, SMADs, Rel-A (p65-NFKB), CREB, NFAT, ATF-2, AFT, Myc, Fos, Sp1, Egr-1, T-bet, .beta.-catenin, HIFs, FOXOs, E2Fs, SRFs, TCFs, Egr-1, \tilde{.beta.}-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

Generally, the methods of the invention involve determining the activation levels of an activatable element in a plurality of single cells in a sample. The activation levels can be obtained by perturbing the cell state using a modulator.

### Signaling Pathways

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been described. See (Hunter T. Cell Jan. 7, 2000; 100(1): 13-27). Exemplary signaling pathways include the following pathways and their members: The MAP kinase pathway including Ras, Raf, MEK, ERK and elk; the PI3K/Akt pathway including PI-3-kinase, PDK1, Akt and Bad; the NF-.kappa.B pathway including IKKs, IkB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, RAS/RAF/MEK/ERK, JNK/SAPK, p38 MAPK, Src Family Kinases, JAK/STAT and/or PKC signaling pathways.

In some embodiments, the methods of the invention are employed to determine the status of a signaling protein in a signaling pathway known in the art including those described herein. Exemplary types of signaling proteins within the scope of the present invention include, but are not limited to kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279).

Nuclear Factor-kappaB (NF-κB) Pathway: Nuclear factor-kappaB (NF-κB) transcription factors and the signaling pathways that activate them are central coordinators of innate and adaptive immune responses. More recently, it has become clear that NF-κB signaling also has a critical role in cancer development and progression. NF-κB provides a mechanistic link between inflammation and cancer, and is a major factor controlling the ability of both pre-neoplastic and malignant cells to resist apoptosis-based tumor-surveillance mechanisms. In mammalian cells, there are five NF-κB family members, RelA (p65), RelB, c-Rel, p50/p105 (NF-κB1) and p52/p 100 (NF-.kappa.B2) and different NF-κB complexes are formed from their homo and heterodimers. In most cell types, NF-κB complexes are retained in the cytoplasm by a family of inhibitory proteins known as inhibitors of NF-κB (I.kappa.Bs). Activation of NF-.kappa.B typically involves the phosphorylation of I.kappa.B by the I.kappa.B kinase (IKK) complex, which results in I.kappa.B ubiquitination with subsequent degradation. This releases NF-κB and allows it to translocate freely to the nucleus. The genes regulated by NF-κB include those controlling programmed cell death, cell adhesion, proliferation, the innate- and adaptive-immune responses, inflammation, the cellular-stress response and tissue remodeling. However, the expression of these genes is tightly coordinated with the activity of many other signaling and transcription-factor pathways. Therefore, the outcome of NF-κB activation depends on the nature and the cellular context of its induction. For example, it has become apparent that NF-κB activity can be regulated by both oncogenes and tumor suppressors, resulting in either stimulation or inhibition of apoptosis and proliferation. See Perkins, N. Integrating cell-signaling pathways with NF-κB and IKK function. Reviews: Molecular Cell Biology. January, 2007; 8(1): 49-62, hereby fully incorporated by reference in its entirety for all purposes. Hayden, M. Signaling to NF-κB. Genes & Development. 2004; 18: 2195-2224, hereby fully incorporated by reference in its entirety for all purposes. Perkins, N. Good Cop, Bad Cop: The Different Faces of NF-κB. Cell Death and Differentiation. 2006; 13: 759-772, hereby fully incorporated by reference in its entirety for all purposes.

Phosphatidylinositol 3-kinase (PI3-K)/AKT Pathway: PI3-Ks are activated by a wide range of cell surface receptors to generate the lipid second messengers phosphatidylinositol 3,4-biphosphate (PIP₂) and phosphatidylinositol 3,4,5-trisphosphate (PIP_{S}). Examples of receptor tyrosine kinases include but are not limited to FLT3 LIGAND, EGFR, IGF-1R, HER2/neu, VEGFR, and PDGFR. The lipid second messengers generated by PI3Ks regulate a diverse array of cellular functions. The specific binding of PI3,4P₂ and PI3,4,5P₃ to target proteins is mediated through the pleckstrin homology (PH) domain present in these target proteins. One key downstream effector of PI3-K is Akt, a serine/threonine kinase, which is activated when its PH domain interacts with PI3,4P₂ and PI3,4,5P₃ resulting in recruitment of Akt to the plasma membrane. Once there, in order to be fully activated, Akt is phosphorylated at threonine 308 by 3-phosphoinositide-dependent protein kinase-1 (PDK-1) and at serine 473 by several PDK2 kinases. Akt then acts downstream of PI3K to regulate the phosphorylation of a number of substrates, including but not limited to forkhead box O transcription factors, Bad, GSK-3β, IκB, mTOR, MDM-2, and S6 ribosomal subunit. These phosphorylation events in turn mediate cell survival, cell proliferation, membrane trafficking, glucose homeostasis, metabolism and cell motility. Deregulation of the PI3K pathway occurs by activating mutations in growth factor receptors, activating mutations in a PI3-K gene (e.g. PIK3CA), loss of function mutations in a lipid phosphatase (e.g. PTEN), up-regulation of Akt, or the impairment of the tuberous sclerosis complex (TSC1/2). All these events are linked to increased survival and proliferation. See Vivanco, I. The Phosphatidylinositol 3-Kinase-AKT Pathway in Human Cancer. Nature Reviews: Cancer. July, 2002; 2: 489-501 and Shaw, R. Ras, PI(3)K and mTOR signaling controls tumor cell growth. Nature. May, 2006; 441: 424-430, Marone et al., Biochimica et Biophysica Acta, 2008; 1784, p159-185 hereby fully incorporated by reference in their entirety for all purposes.

Wnt Pathway: The Wnt signaling pathway describes a complex network of proteins well known for their roles in embryogenesis, normal physiological processes in adult animals, such as tissue homeostasis, and cancer. Further, a role for the Wnt pathway has been shown in self-renewal of hematopoietic stem cells (Reya T et al., Nature. 2003 May 22; 423(6938):409-14). Cytoplasmic levels of .beta.-catenin are normally kept low through the continuous proteosomal degradation of .beta.-catenin controlled by a complex of glycogen synthase kinase 3.beta. (GSK-3.β axin, and adenomatous polyposis coli (APC). When Wnt proteins bind to a receptor complex composed of the Frizzled receptors (Fz) and low density lipoprotein receptor-related protein (LRP) at the cell surface, the GSK-3/axin/APC complex is inhibited. Key intermediates in this process include disheveled (Dsh) and axin binding the cytoplasmic tail of LRP. Upon Wnt signaling and inhibition of the .beta.-catenin degradation pathway, .beta.-catenin accumulates in the cytoplasm and nucleus. Nuclear .beta.-catenin interacts with transcription factors such as lymphoid enhanced-binding factor 1 (LEF) and T cell-specific transcription factor (TCF) to affect transcription of target genes. See Gordon, M. Wnt Signaling: Multiple Pathways, Multiple Receptors, and Multiple Transcription Factors. J of Biological Chemistry. June, 2006; 281(32): 22429-22433, Logan C Y, Nusse R: The Wnt signaling pathway in development and disease. Annu Rev Cell Dev Biol 2004, 20:781-810, Clevers H: Wnt/beta-catenin signaling in development and disease. Cell 2006, 127:469-480. hereby fully incorporated by reference in its entirety for all purposes.

Protein Kinase C(PKC) Signaling: The PKC family of serine/threonine kinases mediate signaling pathways following activation of receptor tyrosine kinases, G-protein coupled receptors and cytoplasmic tyrosine kinases. Activation of PKC family members is associated with cell proliferation, differentiation, survival, immune function, invasion, migration and angiogenesis. Disruption of PKC signaling has been implicated in tumorigenesis and drug resistance. PKC isoforms have distinct and overlapping roles in cellular functions. PKC was originally identified as a phospholipid and calcium-dependent protein kinase. The mammalian PKC superfamily consists of 13 different isoforms that are divided into four subgroups on the basis of their structural differences and related cofactor requirements cPKC (classical PKC) isoforms (.alpha., .beta.I, .beta.II and .gamma.), which respond both to Ca2+ and DAG (diacylglycerol), nPKC (novel PKC) isoforms (.delta., .epsilon., .theta. and .eta.), which are insensitive to Ca2+, but dependent on DAG, atypical PKCs (aPKCs, .dwnarw./.lamda., .zeta.), which are responsive to neither co-factor, but may be activated by other lipids and through protein-protein interactions, and the related PKN (protein kinase N) family (e.g. PKN1, PKN2 and PKN3), members of which are subject to regulation by small GTPases. Consistent with their different biological functions, PKC isoforms differ in their structure, tissue distribution, subcellular localization, mode of activation and substrate specificity. Before maximal activation of its kinase, PKC requires a priming phosphorylation which is provided constitutively by phosphoinositide-dependent kinase 1 (PDK-1). The phospholipid DAG has a central role in the activation of PKC by causing an increase in the affinity of classical PKCs for cell membranes accompanied by PKC activation and the release of an inhibitory substrate (a pseudo-substrate) to which the inactive enzyme binds. Activated PKC then phosphorylates and activates a range of kinases. The downstream events following PKC activation are poorly understood, although the MEK-ERK (mitogen activated protein kinase kinase-extracellular signal-regulated kinase) pathway is thought to have an important role. There is also evidence to support the involvement of PKC in the PI3K-Akt pathway. PKC isoforms probably form part of the multi-protein complexes that facilitate cellular signal transduction. Many reports describe dysregulation of several family members. For example alterations in PKC have been detected in thyroid cancer, and have been correlated with aggressive, metastatic breast cancer and PKC was shown to be associated with poor outcome in ovarian cancer. (Knauf J A, et al. Isozyme-Specific Abnormalities of PKC in Thyroid Cancer Evidence for Post-Transcriptional Changes in PKC Epsilon. The Journal of Clinical Endocrinology & Metabolism. Vol. 87, No. 5, pp 2150-2159; Zhang L et al. Integrative Genomic Analysis of Protein Kinase C (PKC) Family Identifies PKC as a Biomarker and Potential Oncogene in Ovarian Carcinoma. Cancer Res. 2006, Vol 66, No. 9, pp 4627-4635).

Mitogen Activated Protein (MAP) Kinase Pathways: MAP kinases transduce signals that are involved in a multitude of cellular pathways and functions in response to a variety of ligands and cell stimuli. (Lawrence et al., Cell Research (2008) 18: 436-442). Signaling by MAPKs affects specific events such as the activity or localization of individual proteins, transcription of genes, and increased cell cycle entry, and promotes changes that orchestrate complex processes such as embryogenesis and differentiation. Aberrant or inappropriate functions of MAPKs have now been identified in diseases ranging from cancer to inflammatory disease to obesity and diabetes. MAPKs are activated by protein kinase cascades consisting of three or more protein kinases in series: MAPK kinase kinases (MAP3Ks) activate MAPK kinases (MAP2Ks) by dual phosphorylation on S/T residues; MAP2Ks then activate MAPKs by dual phosphorylation on Y and T residues MAPKs then phosphorylate target substrates on select S/T residues typically followed by a proline residue. In the ERK1/2 cascade the MAP3K is usually a member of the Raf family. Many diverse MAP3Ks reside upstream of the p38 and the c-Jun N-terminal kinase/stress-activated protein kinase (JNK/SAPK) MAPK groups, which have generally been associated with responses to cellular stress. Downstream of the activating stimuli, the kinase cascades may themselves be stimulated by combinations of small G proteins, MAP4Ks, scaffolds, or oligomerization of the MAP3K in a pathway. In the ERK1/2 pathway, Ras family members usually bind to Raf proteins leading to their activation as well as to the subsequent activation of other downstream members of the pathway.

Ras/RAF/MEK/ERK Pathway: Classic activation of the RAS/Raf/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MAPK/Erk kinase (MEK)/Erk MAPK pathway and other MAPK pathways as well as many other signaling cascades. The pivotal role of the Ras/Raf/MEK/Erk MAPK pathway in multiple cellular functions underlies the importance of the cascade in oncogenesis and growth of transformed cells. As such, the MAPK pathway has been a focus of intense investigation for therapeutic targeting. Many receptor tyrosine kinases are capable of initiating MAPK signaling. They do so after activating phosphorylation events within their cytoplasmic domains provide docking sites for src-homology 2 (SH2) domain-containing signaling molecules. Of these, adaptor proteins such as Grb2 recruit guanine nucleotide exchange factors such as SOS-1 or CDC25 to the cell membrane. The guanine nucleotide exchange factor is now capable of interacting with Ras proteins at the cell membrane to promote a conformational change and the exchange of GDP for GTP bound to Ras. Multiple Ras isoforms have been described, including K-Ras, N-Ras, and H-Ras. Termination of Ras activation occurs upon hydrolysis of RasGTP to RasGDP. Ras proteins have intrinsically low GTPase activity. Thus, the GTPase activity is stimulated by GTPase-activating proteins such as NF-1 GTPase-activating protein/neurofibromin and p120 GTPase activating protein thereby preventing prolonged Ras stimulated signaling. Ras activation is the first step in activation of the MAPK cascade. Following Ras activation, Raf (A-Raf, B-Raf, or Raf-1) is recruited to the cell membrane through binding to Ras and activated in a complex process involving phosphorylation and multiple cofactors that is not completely understood. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation. Although MEK1/2 have no known targets besides Erk proteins, Erk has multiple targets including Elk-1, c-Ets1, c-Ets2, p90RSK1, MNK1, MNK2, and TOB. The cellular functions of Erk are diverse and include regulation of cell proliferation, survival, mitosis, and migration. McCubrey, J. Roles of the Raf/MEK/ERK pathway in cell growth, malignant transformation and drug resistance. Biochimica et Biophysica Acta. 2007; 1773: 1263-1284, hereby fully incorporated by reference in its entirety for all purposes, Friday and Adjei, Clinical Cancer Research (2008) 14, p342-346.

c-Jun N-terminal kinase (INK)/stress-activated protein kinase (SAPK) Pathway: The c-Jun N-terminal kinases (JNKs) were initially described as a family of serine/threonine protein kinases, activated by a range of stress stimuli and able to phosphorylate the N-terminal transactivation domain of the c-Jun transcription factor. This phosphorylation enhances c-Jun dependent transcriptional events in mammalian cells. Further research has revealed three JNK genes (JNK1, JNK2 and JNK3) and their splice-forms as well as the range of external stimuli that lead to JNK activation. JNK1 and JNK2 are ubiquitous, whereas JNK3 is relatively restricted to brain. The predominant MAP2Ks upstream of JNK are MEK4 (MKK4) and MEK7 (MKK7). MAP3Ks with the capacity to activate JNK/SAPKs include MEKKs (MEKK1, -2, -3 and -4), mixed lineage kinases (MLKs, including MLK1-3 and DLK), Tp12, ASKs, TAOs and TAK1. Knockout studies in several organisms indicate that different MAP3Ks predominate in JNK/SAPK activation in response to different upstream stimuli. The wiring may be comparable to, but perhaps even more complex than, MAP3K selection and control of the ERK1/2 pathway. JNK/SAPKs are activated in response to inflammatory cytokines; environmental stresses, such as heat shock, ionizing radiation, oxidant stress and DNA damage; DNA and protein synthesis inhibition; and growth factors. JNKs phosphorylate transcription factors c-Jun, ATF-2, p53, Elk-1, and nuclear factor of activated T cells (NFAT), which in turn regulate the expression of specific sets of genes to mediate cell proliferation, differentiation or apoptosis. JNK proteins are involved in cytokine production, the inflammatory response, stress-induced and developmentally programmed apoptosis, actin reorganization, cell transformation and metabolism. Raman, M. Differential regulation and properties of MAPKs. Oncogene. 2007; 26: 3100-3112, hereby fully incorporated by reference in its entirety for all purposes.

p38 MAPK Pathway: Several independent groups identified the p38 Map kinases, and four p38 family members have been described. Although the p38 isoforms share about 40% sequence identity with other MAPKs, they share only about 60% identity among themselves, suggesting highly diverse functions. p38 MAPKs respond to a wide range of extracellular cues particularly cellular stressors such as UV radiation, osmotic shock, hypoxia, pro-inflammatory cytokines and less often growth factors. Responding to osmotic shock might be viewed as one of the oldest functions of this pathway, because yeast p38 activates both short and long-term homeostatic mechanisms to osmotic stress. p38 is activated via dual phosphorylation on the TGY motif within its activation loop by its upstream protein kinases MEK3 and MEK6. MEK3/6 are activated by numerous MAP3Ks including MEKK1-4, TAOs, TAK and ASK. p38 MAPK is generally considered to be the most promising MAPK therapeutic target for rheumatoid arthritis as p38 MAPK isoforms have been implicated in the regulation of many of the processes, such as migration and accumulation of leucocytes, production of cytokines and pro-inflammatory mediators and angiogenesis, that promote disease pathogenesis. Further, the p38 MAPK pathway plays a role in cancer, heart and neurodegenerative diseases and may serve as promising therapeutic target. Cuenda, A. p38 MAP-Kinases pathway regulation, function, and role in human diseases. Biochimica et Biophysica Acta. 2007; 1773: 1358-1375; Thalhamer et al., Rheumatology 2008; 47:409-414; Roux, P. ERK and p38 MAPK-Activated Protein Kinases: a Family of Protein Kinases with Diverse Biological Functions. Microbiology and Molecular Biology Reviews. June, 2004; 320-344 hereby fully incorporated by reference in its entirety for all purposes.

Src Family Kinases: Src is the most widely studied member of the largest family of nonreceptor protein tyrosine kinases, known as the Src family kinases (SFKs). Other SFK members include Lyn, Fyn, Lck, Hck, Fgr, Blk, Yrk, and Yes. The Src kinases can be grouped into two sub-categories, those that are ubiquitously expressed (Src, Fyn, and Yes), and those which are found primarily in hematopoietic cells (Lyn, Lck, Hck, Blk, Fgr). (Benati, D. Src Family Kinases as Potential Therapeutic Targets for Malignancies and Immunological Disorders. Current Medicinal Chemistry. 2008; 15: 1154-1165) SFKs are key messengers in many cellular pathways, including those involved in regulating proliferation, differentiation, survival, motility, and angiogenesis. The activity of SFKs is highly regulated intramolecularly by interactions between the SH2 and SH3 domains and intermolecularly by association with cytoplasmic molecules. This latter activation may be mediated by focal adhesion kinase (FAK) or its molecular partner Crk-associated substrate (CAS), which play a prominent role in integrin signaling, and by ligand activation of cell surface receptors, e.g. epidermal growth factor receptor (EGFR). These interactions disrupt intramolecular interactions within Src, leading to an open conformation that enables the protein to interact with potential substrates and downstream signaling molecules. Src can also be activated by dephosphorylation of tyrosine residue Y530. Maximal Src activation requires the autophosphorylation of tyrosine residue Y419 (in the human protein) present within the catalytic domain. Elevated Src activity may be caused by increased transcription or by deregulation due to overexpression of upstream growth factor receptors such as EGFR, HER2, platelet-derived growth factor receptor (PDGFR), fibroblast growth factor receptor (FGFR), vascular endothelial growth factor receptor, ephrins, integrin, or FAK. Alternatively, some human tumors show reduced expression of the negative Src regulator, Csk. Increased levels, increased activity, and genetic abnormalities of Src kinases have been implicated in both solid tumor development and leukemias. Ingley, E. Src family kinases: Regulation of their activities, levels and identification of new pathways. Biochimica et Biophysica Acta. 2008; 1784 56-65, hereby fully incorporated by reference in its entirety for all purposes. Benati and Baldari., Curr Med. Chem. 2008; 15(12):1154-65, Finn (2008) Ann Oncol. May 16, hereby fully incorporated by reference in its entirety for all purposes.

Janus kinase (JAK)/Signal transducers and activators of transcription (STAT) pathway: The JAK/STAT pathway plays a crucial role in mediating the signals from a diverse spectrum of cytokine receptors, growth factor receptors, and G-protein-coupled receptors. Signal transducers and activators of transcription (STAT) proteins play a crucial role in mediating the signals from a diverse spectrum of cytokine receptors growth factor receptors, and G-protein-coupled receptors. STAT directly links cytokine receptor stimulation to gene transcription by acting as both a cytosolic messenger and nuclear transcription factor. In the Janus Kinase (JAK)-STAT pathway, receptor dimerization by ligand binding results in JAK family kinase (JFK) activation and subsequent tyrosine phosphorylation of the receptor, which leads to the recruitment of STAT through the SH2 domain, and the phosphorylation of conserved tyrosine residue. Tyrosine phosphorylated STAT forms a dimer, translocates to the nucleus, and binds to specific DNA elements to activate target gene transcription, which leads to the regulation of cellular proliferation, differentiation, and apoptosis. The entire process is tightly regulated at multiple levels by protein tyrosine phosphatases, suppressors of cytokine signaling and protein inhibitors of activated STAT. In mammals seven members of the STAT family (STAT1, STAT2, STAT3, STAT4, STAT5a, STAT5b and STATE) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3. Mutations in JAK proteins have been described for several myeloid malignancies. Specific examples include but are not limited to: Somatic JAK3 (e.g. JAK3A572V, JAK3V722I, JAK3P132T) and fusion JAK2 (e.g. ETV6-JAK2, PCM1-JAK2, BCR-JAK2) mutations have respectively been described in acute megakaryocytic leukemia and acute leukemia/chronic myeloid malignancies, JAK2 (V617F, JAK2 exon 12 mutations) and MPL MPLW515L/K/S, MPLS505N) mutations associated with myeloproliferative disorders and myeloproliferative neoplasms. JAK2 mutations, primarily JAK2V617F, are invariably associated with polycythemia vera (PV). This mutation also occurs in the majority of patients with essential thrombocythemia (ET) or primary myelofibrosis (PMF) (Tefferi n., Leukemia & Lymphoma, March 2008; 49(3): 388-397). STATs can be activated in a JAK-independent manner by src family kinase members and by oncogenic FLt3 ligand-ITD (Hayakawa and Naoe, Ann NY Acad. Sci. 2006 November; 1086:213-22; Choudhary et al. Activation mechanisms of STAT5 by oncogenic FLt3 ligand-ITD. Blood (2007) vol. 110 (1) pp. 370-4). Although mutations of STATs have not been described in human tumors, the activity of several members of the family, such as STAT1, STAT3 and STAT5, is dysregulated in a variety of human tumors and leukemias. STAT3 and STAT5 acquire oncogenic potential through constitutive phosphorylation on tyrosine, and their activity has been shown to be required to sustain a transformed phenotype. This was shown in lung cancer where tyrosine phosphorylation of STAT3 was JAK-independent and mediated by EGF receptor activated through mutation and Src. (Alvarez et al., Cancer Research, Cancer Res 2006; 66) STAT5 phosphorylation was also shown to be required for the long-term maintenance of leukemic stem cells. (Schepers et al. STAT5 is required for long-term maintenance of normal and leukemic human stem/progenitor cells. Blood (2007) vol. 110 (8) pp. 2880-2888) In contrast to STAT3 and STAT5, STAT1 negatively regulates cell proliferation and angiogenesis and thereby inhibits tumor formation. Consistent with its tumor suppressive properties, STAT1 and its downstream targets have been shown to be reduced in a variety of human tumors (Rawlings, J. The JAK/STAT signaling pathway. J of Cell Science. 2004; 117 (8):1281-1283, hereby fully incorporated by reference in its entirety for all purposes).

### Drug Transporters

In some embodiments, the present invention provides methods for classification, diagnosis, prognosis of a condition and/or prediction of outcome after administering a therapeutic agent to treat the condition by determining a drug transporter expression and/or function. In some embodiments, the present invention provides methods for classification, diagnosis, prognosis of disease and/or prediction of outcome after administering a therapeutic agent to treat the condition by determining a drug transporter expression and/or function and by characterizing one or more pathways in a population of cells. In some embodiments, the therapeutic agent is a drug transporter substrate.

A key issue in the treatment of many cancers is the development of resistance to chemotherapeutic drugs. Of the many resistance mechanisms, two classes of transporters play a major role. The human ATP-binding cassette (ABC) superfamily of proteins consists of 49 membrane proteins that transport a diverse array of substrates, including sugars, amino acids, bile salts lipids, sterols, nucleotides, endogenous metabolites, ions, antibiotics drugs and toxins out of cells using the energy of hydrolysis of ATP. ATP-binding-cassette (ABC) transporters are evolutionary extremely well-conserved transmembrane proteins that are highly expressed in hematopoietic stem cells (HSCs). The physiological function in human stem cells is believed to be protection against genetic damage caused by both environmental and naturally occurring xenobiotics. Additionally, ABC transporters have been implicated in the maintenance of quiescence and cell fate decisions of stem cells. These physiological roles suggest a potential role in the pathogenesis and biology of stem cell-derived hematological malignancies such as acute and chronic myeloid leukemia (Raaijmakers, Leukemia (2007) 21, 2094-2102, Zhou et al., Nature Medicine, 2001, 7, p 1028-1034

Several ABC proteins are multidrug efflux pumps that not only protect the body from exogenous toxins, but also play a role in uptake and distribution of therapeutic drugs. Expression of these proteins in target tissues causes resistance to treatment with multiple drugs. (Gillet et al., Biochimica et Biophysica Acta (2007) 1775, p 237, Sharom (2008) Pharmacogenomics 9 p 105). A more detailed discussion of the ABC family members with critical roles in resistance and poor outcome to treatment is discussed below.

The second class of plasma membrane transporter proteins that play a role in the uptake of nucleoside-derived drugs are the Concentrative and Equilibrative Nucleoside Transporters (CNT and ENT, respectively), encoded by gene families SLC28 and SLC29 (Pastor-Anglada (2007) J. Physiol. Biochem 63, p 97). They mediate the uptake of natural nucleosides and a variety of nucleoside-derived drugs, mostly used in anti-cancer therapy. In vitro studies, have shown that one mechanism of nucleoside resistance can be mediated through mutations in the gene for ENT1/SLC29A1 resulting in lack of detectable protein (Cai et al., Cancer Research (2008) 68, p 2349). Studies have also described in vivo mechanisms of resistance to nucleoside analogues involving low or non-detectable levels of ENT1 in Acute Myeloid Leukemia (AML), Mantle Cell lymphoma and other leukemias (Marce et al., Malignant Lymphomas (2006), 91, p 895).

Of the ABC transporter family, three family members account for most of the multiple drug resistance (MDR) in humans; P-glycoprotein (Pgp/MDR1/ABCB1), MDR associated protein (MRP1, ABCC1) and breast cancer resistance protein (BCRP, ABCG2 or MXR). Pgp/MDR1 and ABCG2 can export both unmodified drugs and drug conjugates, whereas MRP1 exports glutathione and other drug conjugates as well as unconjugated drugs together with free glutathione. All three ABC transporters demonstrate export activity for a broad range of structurally unrelated drugs and display both distinct and overlapping specificities. For example, MRP1 promotes efflux of drug-glutathione conjugates, vinca alkaloids, camptothecin, but not taxol. Examples of drugs exported by ABCG2 include mitoxantrone, etoposide, daunorubicin as well as the tyrosine kinase inhibitors Gleevec and Iressa. In treatment regimens for leukemias, one of the main obstacles to achieving remission is intrinsic and acquired resistance to chemotherapy mediated by the ABC drug transporters. Several reports have described correlations between transporter expression levels as well as their function, evaluated through the use of fluorescent dyes, with resistance of patients to chemotherapy regimens. Notably, in AML, studies have shown that expression of Pgp/MDR1 is associated with a lower rate of complete response to induction chemotherapy and a higher rate of resistant disease in both elderly and younger AML patients (Leith et al., Blood (1997) 89 p 3323, Leith et al., Blood (1999) 94, p 1086). Legrand et al., (Blood (1998) 91, p 4480) showed that Pgp/MDR1 and MRP1 function in CD34+ blast cells are negative prognostic factors in AML and further, the same group showed that a high level of simultaneous activity of Pgp/MDR1 and MRP1 was predictive of poor treatment outcome (Legrand et al., (Blood (1999) 94, p 1046). In two more recent studies, elevated expression of Pgp/MDR1 and BCRP in CD34+/CD38− AML subpopulations were found in 8 out of 10 non-responders as compared to 0 out of 10 in responders to induction chemotherapy (Ho et al., Experimental Hematology (2008) 36, p 433). In a second study, evaluation of Pgp/MDR1, MRP1, BCRP/ABCG2 and lung resistance protein showed that the more immature subsets of leukemic stem cells expressed higher levels of these proteins compared more mature leukemic subsets (Figueiredo-Pontes et al., Clinical Cytometry (2008) 74B p 163).

Experimentally, it is possible to correlate expression of transporter proteins with their function by the use of inhibitors including but not limited to cyclosporine (measures Pgp function), probenecid (measures MRP1 function), fumitremorgin C, and a derivative Kol43, reserpine (measures ABCG2 function). Although these molecules inhibit a variety of transporters, they do permit some correlations to be made between protein expression and function (Legrand et al., (Blood (1998) 91, p 4480), Legrand et al., (Blood (1999) 94, p 1046, Zhou et al., Nature Medicine, 2001, 7, p 1028-1034, Sarkardi et al., Physiol Rev 2006 86: 1179-1236).

Extending the use of these inhibitors, they can be used to make correlations within subpopulations of cells gated both for phenotypic markers denoting stages of development along hematopoietic and lymphoid lineages, as well as reagents that recognize the transporter proteins themselves. Thus it will be possible to simultaneously measure protein expression and function

### DNA Damage and Apoptosis

The response to DNA damage is a protective measure taken by cells to prevent or delay genetic instability and tumorigenesis. It allows cells to undergo cell cycle arrest and gives them an opportunity to either: repair the damaged DNA and resume passage through the cell cycle or, if the damage is irreparable, trigger senescence or an apoptotic program leading to cell death (Wade Harper et al., Molecular Cell, (2007) 28 p 739.+−.745, Bartek J et al., Oncogene (2007)26 p 7773-9).

Several protein complexes are positioned at strategic points within the DNA damage response pathway and act as sensors of DNA damage, or transducers or effectors of a DNA damage response. Depending on the nature of DNA damage for example; double stranded breaks, single strand breaks, single base alterations due to alkylation, oxidation etc, there is an assembly of specific DNA damage sensor protein complexes in which activated ataxia telangiectasia mutated (ATM) and ATM- and Rad3 related (ATR) kinases phosphorylate and subsequently activate the checkpoint kinases Chk1 and Chk2. Both of these DNA-signal transducer kinases amplify the damage response by phosphorylating a multitude of substrates. Both checkpoint kinases have overlapping and distinct roles in orchestrating the cell's response to DNA damage.

Activation of Chk2 kinase activity involves ATM mediated phosphorylation of threonine 68 and homo-dimerization (Reinhardt H C, Yaffe M B Curr Opin Cell Biol. 2009 April; 21(2):245-55, Antoni L, Sodha N, Collins I, Garrett M D Nat Rev Cancer. 2007 December; 7(12):925-36. This in turn initiates the DNA repair process of which there are at least twelve distinct mechanisms. The choice of which repair process to use depends on the type of lesion and on the cell-cycle phase of the cell. For example, a DNA double-strand break (DSB) in S and G2 phases is readily repaired by homologous recombination (Branzei and Foiani Nat Rev Mol Cell Biol. 2008 April; 9(4):297-308). If DNA repair is successful cell cycle progression is resumed (Antoni et al., Nature reviews cancer (2007) 7, p 925-936).

When DNA repair is no longer possible, the cell undergoes apoptosis mediated by Chk-2 through p53 independent and dependent pathways. Chk2 substrates that operate in a p53-independent manner include the E2F1 transcription factor, the tumor suppressor promyelocytic leukemia (PML) and the polo-like kinases 1 and 3 (PLK1 and PLK3). E2F1 drives the expression of a number of apoptotic genes including caspases 3, 7, 8 and 9 as well as the pro-apoptotic Bcl-2 related proteins (Bim, Noxa, PUMA).

In its response to DNA damage, p53 activates the transcription of a program of genes that regulate DNA repair, cell cycle arrest, senescence and apoptosis. The overall functions of p53 are to preserve fidelity in DNA replication such that when cell division occurs tumorigenic potential can be avoided. In such a role, p53 is described as “The Guardian of the Genome (Riley et al., Nature Reviews Molecular Cell Biology (2008) 9 p402-412). The diverse alarm signals that impinge on p53 result in a rapid increase in its levels through a variety of post translational modifications. Worthy of mention is the phosphorylation of amino acid residues within the amino terminal portion of p53 such that p53 is no longer under the regulation of Mdm2 The responsible kinases are ATM, Chk1 and Chk2. The subsequent stabilization of p53 permits it to transcriptionally regulate multiple pro-apoptotic members of the Bcl-2 family, including Bax, Bid, Puma, and Noxa (Discussion below).

The series of events that are mediated by p53 to promote apoptosis including DNA damage, anoxia and imbalances in growth-promoting signals are sometimes termed the “intrinsic apoptotic” program since the signals triggering it originate within the cell. An alternate route of activating the apoptotic pathway can occur from the outside of the cell mediated by the binding of ligands to transmembrane death receptors. This extrinsic or receptor mediated apoptotic program acting through their receptor death domains eventually converges on the intrinsic, mitochondrial apoptotic pathway as discussed below (Sprick et al., Biochim Biophys Acta. (2004) 1644 p 125-32).

Key regulators of apoptosis are proteins of the Bcl-2 family. The founding member, the Bcl-2 proto-oncogene was first identified at the chromosomal breakpoint of t(14:18) bearing human follicular B cell lymphoma. Unexpectedly, expression of Bcl-2 was proved to block rather than promote cell death following multiple pathological and physiological stimuli (Danial and Korsemeyer, Cell (2204) 116, p 205-219). The Bcl-2 family has at least 20 members which are key regulators of apoptosis, functioning to control mitochondrial permeability as well as the release of proteins important in the apoptotic program. The ratio of anti- to pro-apoptotic molecules such as Bcl-2/Bax constitutes a rheostat that sets the threshold of susceptibility to apoptosis for the intrinsic pathway, which utilizes organelles such as the mitochondrion to amplify death signals. The family can be divided into 3 subclasses based on structure and impact on apoptosis. Family members of subclass 1 including Bcl-2, Bcl-X_{L} and Mcl-1 are characterized by the presence of 4 Bcl-2 homology domains (BH1, BH2, BH3 and BH4) and are anti-apoptotic. The structure of the second subclass members is marked for containing 3 BH domains and family members such as Bax and Bak possess pro-apoptotic activities. The third subclass, termed the BH3-only proteins include Noxa, Puma, Bid, Bad and Bim. They function to promote apoptosis either by activating the pro-apoptotic members of group 2 or by inhibiting the anti-apoptotic members of subclass 1 (Er et al., Biochimica et Biophysica Act (2006) 1757, p 1301-1311, Fernandez-Luna Cellular Signaling (2008) Advance Publication Online).

The role of mitochondria in the apoptotic process was clarified as involving an apoptotic stimulus resulting in depolarization of the outer mitochondrial membrane leading to a leak of cytochrome C into the cytoplasm. Association of cytochrome C molecules with adaptor apoptotic protease activating factor (APAF) forms a structure called the apoptosome which can activate enzymatically latent procaspase 9 into a cleaved activated form. Caspase 9 is one member of a family of cysteine aspartyl-specific proteases; genes encoding 11 of these proteases have been mapped in the human genome. Activated caspase 9, classified as an intiator caspase, then cleaves procaspase 3 which cleaves more downstream procaspases, classified as executioner caspases, resulting in an amplification cascade that promotes cleavage of death substrates including poly(ADP-ribose) polymerase 1 (PARP). The cleavage of PARP produces 2 fragments both of which have a role in apoptosis (Soldani and Scovassi Apoptosis (2002) 7, p 321). A further level of apoptotic regulation is provided by smac/Diablo, a mitochondrial protein that inactivates a group of anti-apoptotic proteins termed inhibitors of apoptosis (IAPs) (Huang et al., Cancer Cell (2004) 5 p 1-2). IAPs operate to block caspase activity in 2 ways; they bind directly to and inhibit caspase activity and in certain cases they can mark caspases for ubiquitination and degradation.

The balance of pro- and anti-apoptotic proteins is tightly regulated under normal physiological conditions. Tipping of this balance either way results in disease. An oncogenic outcome results from the inability of tumor cells to undergo apoptosis and this can be caused by over-expression of anti-apoptotic proteins or reduced expression or activity of pro-apoptotic protein.

### Cell Cycle

The cell cycle, or cell-division cycle, is the series of events that take place in a cell leading to its division and duplication (replication). The cell cycle consists of five distinct phases: G0 phase, G1 phase, S (synthesis) phase, G2 phase (these four phases are collectively known as interphase) and M phase (mitosis). M phase is itself composed of two tightly coupled processes: mitosis, in which the cell's chromosomes are divided between two daughter cells, and cytokinesis, in which the cell's cytoplasm divides forming distinct cells. Activation of each of the five cell cycle phases is dependent on the proper progression and completion of the previous one. Cells that have temporarily or reversibly stopped dividing are said to have entered a state of quiescence called G0 phase.

Regulation of the cell cycle involves processes crucial to the survival of a cell, including the detection and repair of genetic damage as well as the prevention of uncontrolled cell division. The molecular events that control the cell cycle are ordered and directional; that is, each process occurs in a sequential fashion and it is impossible to “reverse” the cycle.

Two key classes of regulatory molecules, cyclins and cyclin-dependent kinases (CDKs), determine a cell's progress through the cell cycle. Many of the genes encoding cyclins and CDKs are conserved among all eukaryotes, but in general more complex organisms have more elaborate cell cycle control systems that incorporate more individual components. Many of the relevant genes were first identified by studying yeast, especially Saccharomyces cerevisiae genetic nomenclature in yeast dubs many these genes cdc (for “cell division cycle”) followed by an identifying number, e.g., cdc25.

Cyclins form the regulatory subunits and CDKs the catalytic subunits of an activated heterodimer; cyclins have no catalytic activity and CDKs are inactive in the absence of a partner cyclin. When activated by a bound cyclin, CDKs perform a common biochemical reaction called phosphorylation that activates or inactivates target proteins to orchestrate coordinated entry into the next phase of the cell cycle. Different cyclin-CDK combinations determine the downstream proteins targeted. CDKs are constitutively expressed in cells whereas cyclins are synthesized at specific stages of the cell cycle, in response to various molecular signals.

Upon receiving a pro-mitotic extracellular signal, G1 cyclin-CDK complexes become active to prepare the cell for S phase, promoting the expression of transcription factors that in turn promote the expression of S cyclins and of enzymes required for DNA replication. The G1 cyclin-CDK complexes also promote the degradation of molecules that function as S phase inhibitors by targeting them for ubiquitination. Once a protein has been ubiquitinated, it is targeted for proteolytic degradation by the proteasome. Active S cyclin-CDK complexes phosphorylate proteins that make up the pre-replication complexes assembled during G1 phase on DNA replication origins. The phosphorylation serves two purposes: to activate each already-assembled pre-replication complex, and to prevent new complexes from forming. This ensures that every portion of the cell's genome will be replicated once and only once. The reason for prevention of gaps in replication is fairly clear, because daughter cells that are missing all or part of crucial genes will die. However, for reasons related to gene copy number effects, possession of extra copies of certain genes would also prove deleterious to the daughter cells.

Mitotic cyclin-CDK complexes, which are synthesized but inactivated during S and G2 phases, promote the initiation of mitosis by stimulating downstream proteins involved in chromosome condensation and mitotic spindle assembly. A critical complex activated during this process is an ubiquitin ligase known as the anaphase-promoting complex (APC), which promotes degradation of structural proteins associated with the chromosomal kinetochore. APC also targets the mitotic cyclins for degradation, ensuring that telophase and cytokinesis can proceed. Interphase: Interphase generally lasts at least 12 to 24 hours in mammalian tissue. During this period, the cell is constantly synthesizing RNA, producing protein and growing in size. By studying molecular events in cells, scientists have determined that interphase can be divided into 4 steps: Gap 0 (G0), Gap 1 (G1), S (synthesis) phase, Gap 2 (G2).

Cyclin D is the first cyclin produced in the cell cycle, in response to extracellular signals (e.g. growth factors). Cyclin D binds to existing CDK4, forming the active cyclin D-CDK4 complex. Cyclin D-CDK4 complex in turn phosphorylates the retinoblastoma susceptibility protein (Rb). The hyperphosphorylated Rb dissociates from the E2F/DP1/Rb complex (which was bound to the E2F responsive genes, effectively “blocking” them from transcription), activating E2F. Activation of E2F results in transcription of various genes like cyclin E, cyclin A, DNA polymerase, thymidine kinase, etc. Cyclin E thus produced binds to CDK2, forming the cyclin E-CDK2 complex, which pushes the cell from G1 to S phase (G1/S transition). Cyclin B along with cdc2 (cdc2-fission yeasts (CDK1-mammalia)) forms the cyclin B-cdc2 complex, which initiates the G2/M transition. Cyclin B-cdc2 complex activation causes breakdown of nuclear envelope and initiation of prophase, and subsequently, its deactivation causes the cell to exit mitosis.

Two families of genes, the Cip/Kip family and the INK4a/ARF (Inhibitor of Kinase 4/Alternative Reading Frame) prevent the progression of the cell cycle. Because these genes are instrumental in prevention of tumor formation, they are known as tumor suppressors.

The Cip/Kip family includes the genes p21, p27 and p57. They halt cell cycle in G1 phase, by binding to, and inactivating, cyclin-CDK complexes. p21 is a p53 response gene (which, in turn, is triggered by DNA damage, e.g. due to radiation). p27 is activated by Transforming Growth Factor .beta. (TGF β), a growth inhibitor.

The INK4a/ARF family includes p161NK4a, which binds to CDK4 and arrests the cell cycle in G1 phase, and p14arf which prevents p53 degradation.

Cell cycle checkpoints are used by the cell to monitor and regulate the progress of the cell cycle. Checkpoints prevent cell cycle progression at specific points, allowing verification of necessary phase processes and repair of DNA damage. The cell cannot proceed to the next phase until checkpoint requirements have been met.

Several checkpoints are designed to ensure that damaged or incompletely synthesized DNA is not passed on to daughter cells. Two main checkpoints exist: the G1/S checkpoint and the G2/M checkpoint. G1/S transition is a rate-limiting step in the cell cycle and is also known as restriction point. An alternative model of the cell cycle response to DNA damage has also been proposed, known as the postreplication checkpoint. p53 plays an important role in triggering the control mechanisms at both G1/S and G2/M checkpoints.

### Modulators

In some embodiments, the methods and composition utilize a modulator. A modulator can be an activator, a therapeutic agent, an inhibitor or a compound capable of impacting a cellular pathway. Modulators can also take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after cells have been isolated. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, chemokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the cross-linker is a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor.

In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

### Detection

In practicing the methods of this invention, the detection of the status of the one or more activatable elements can be carried out by a person, such as a technician in the laboratory or in a clinical setting. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detects and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy in either single cell or high-throughput formats were hundreds of thousands to millions of cells can be counted, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See the above patents and applications for example methods.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorchrome to form a detection element. Detection elements do not need to have both of the above agents, but can be one unit that possesses both qualities. These and other methods are well described in U.S. Pat. Nos. 7,381,535 and 7,393,656 and U.S. Ser. Nos. 10/193,462; 11/655,785; 11/655,789; 11/655,821; 11/338,957, 61/048,886; 61/048,920; and 61/048,657 which are all incorporated by reference in their entireties.

In one embodiment of the invention, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions of the present invention, it will be recognized that different types of fluorescent monitoring systems, e.g., high-content cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response to excitation by the excitation source, the fluorescent molecules that are conjugated to the proteins in the sample can emit a photon following relaxation of the molecule to the lower energy state. The emitted photon has a wavelength that is different and higher than that of the excitation wavelength (e.g. excitation of a fluorescent molecule that accepts exciation at a wavelength of 488 may emit light at a wavelength greater than 500 nm). Collection optics, or a collection pathway including either a laser scanning device or a camera with a collection chip then collect the emission from the sample. The device can include a temperature controller to maintain the sample at a specific temperature while it is being scanned. Additionally, one can include a device to deliver CO₂ in accordance with cellular needs (e.g. human cells normally require 5% CO₂). According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ Training Manual, with particular reference to sections 3-11 to 3-28 and 10-1 to 10-17, which is hereby incorporated by reference in its entirety. See the patents, applications and articles referred to, and incorporated above for detection systems.

Fluorescent compounds such as Daunorubicin and Enzastaurin are problematic for flow cytometry based biological assays due to their broad fluorescence emission spectra. These compounds get trapped inside cells after fixation with agents like paraformaldehyde, and are excited by one or more of the lasers found on flow cytometers. The fluorescence emission of these compounds is often detected in multiple PMT detectors which complicates their use in multiparametric flow cytometry. A way to get around this problem is to compensate out the fluorescence emission of the compound from the PMT detectors used to measure the relevant biological markers. This is achieved using a PMT detector with a bandpass filter near the emission maximum of the fluorescent compound, and cells incubated with the compound as the compensation control when calculating a compensation matrix. The cells incubated with the fluorescent compound are fixed with paraformaldehyde, then washed and permeabilized with 100% methanol. The methanol is washed out and the cells are mixed with unlabeled fixed/permed cells to yield a compensation control consisting of a mixture of fluorescent and negative cell populations.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific binding element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation levels, altered response to modulators) as compared to other subsets within the population. For example, as outlined herein, the methods of the invention allow the identification of subsets of cells from a population such as primary cell populations that exhibit altered responses (e.g. response associated with presence of a condition) as compared to other subsets. The methods of the invention allow the identification of subsets of cells from a population of cells, including, but not limited to used estrogen positive immortalized breast cancer positive cells, MCF7 cells, and HeLa cells. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such as peripheral blood mononuclear cells, or naive and memory lymphocytes.

When necessary cells can be dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells can be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, DNA microarrays are commercially available through a variety of sources (Affymetrix, Santa Clara Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments, confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described elsewhere herein.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated. See U.S. Ser. No. 61/048,657.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution. Both EMCCD and CCD cameras can be used to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory. In some aspects, in order to further to increase analysis speed and accuracy, graphical processing units like those used in gaming systems (Xbox, Play Station) can be used to hold , store and process the large data sets.

These robotic fluid handling systems can utilize any number of different reagents, including buffers, reagents, samples, washes, assay components such as label probes, etc.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

### Antibody Quality and Specificity and Antibody Purification

Clearly, the usefulness of an antibody for immunofluorescence protocols requires a far more stringent assessment of its specificity than for certain other techniques. While irrelevant cross-reacting proteins can often be ignored in Western blotting procedures, assuming that the cross-reacting proteins migrate at distinctly different positions on the SDS gel than the protein of interest, no such cross-reacting material can be tolerated in immunofluorescence images. Many procedures have been developed for the purification of IgG molecules from raw serum samples or mouse ascites fluid that involve affinity purifications or the use of IgG-specific resins containing coupled Staphylococcus protein A or Streptococcal protein G [K. Huse, et al. Biochem. Biophys. Methods 51 (2002)217-231; M. Page, R. Thorpe, In the Protein Protocols Handbook, in: J. M. Walker (Ed.), Humana Press Inc., Totowa, N. J., 2002, pp. 993-994], and numerous protocols exist on various company websites. Many protocols involve elution from the affinity resin using low pH buffers, which can severely reduce antibody quality if the time of exposure is too long. However, it has been found that a modified protein A (Bio-Rad) allows efficient and quantitative elution of IgG from both serum and ascites fluid using buffers with a pH ranging from 4.5 to 6.5. Additionally, to minimize possible damage to the antibody when using buffers at the lower end of this range, elution is done directly into a Tris-based buffer at pH 9.0 resulting in a product that is either at or near physiological pH. For purifications starting with larger volumes of ascites fluid or serum, the samples are divided such that multiple 1-2 ml bed volume columns are used as opposed to larger volume columns, thus reducing the time of exposure of protein to the lower pH. Finally, it has been found to be critical to buffer-exchange and store the purified product in phosphate buffered saline (PBS) to ensure that it is maintained at physiological pH. Also, it was found that if one wishes to preserve the purified antibody for long periods azide can be added in small quantities to the solution, however it should be noted that azide has been shown to be capable of reducing fluorescent signal strength. A Western blot showed purification of anti-HsXrcc3 monoclonal antibody enhances specificity in Western blot procedures. HEK293 whole cell extracts were analyzed by Western blot for the presence of the Xrcc3 protein. Neighboring lanes of a 10% SDS polyacrylamide gel were loaded with 60 lg total protein, and following transfer to a nitrocellulose membrane the lanes were separated. One was incubated with ascites fluid containing anti-HsXrcc antibody and the other was incubated with antibody purified from the ascites fluid. Incubation times with antibody, washes, incubation times with Visualizer™ ECL reagent (Upstate Inc.—now Millipore) and exposure times were identical for both blots. The Western Blot illustrated an example of how purification of a commercially available monoclonal antibody directed against the human Xrcc3 protein (originally obtained from Novus) can offer a significant improvement in antibody specificity. Whereas multiple cross-reacting bands appear when using the raw ascites fluid containing the anti-Xrcc3 antibody, use of the modified protein A purified IgG results in the appearance of a single protein band at the appropriate molecular weight, 37.8 kDa.

For immunofluorescence studies, control experiments demonstrating the exclusive interaction of an antibody with its specific antigen are important. Thus, immunofluorescence work should include the use of purified and well-characterized antibodies. As an example of the problems that can result when using non-purified antibodies, even those that may show only a trace of cross-reactivity. The results showed purification of anti-HsXrcc3 monoclonal antibody enhances specificity and detection of protein by immunofluorescence. HEK293 cells exposed to 8 Gy IR were fixed and stained with either anti-HsXrcc3 mouse ascites fluid or purified anti-HsXrcc3 antibody, followed by staining with a goat anti-mouse Alexa 488 secondary antibody conjugate. Antibody dilutions were 1:500 (primary) and 1:1000 (secondary). Images from these experiments showed a comparison of the use of a non-purified and purified anti-Xrcc3 antibody. HEK293 cells were methanol-fixed (−20° C., 8 min) 2 h following exposure to 8 Gy ionizing radiation (IR), a treatment frequently used to induce DNA damage including DNA double strand breaks (DSB). Cells were stained for Xrcc3 using either the raw ascites fluid or the purified antibody. With the raw ascites fluid signal is observed throughout the entire volume of the cell in a diffuse, seemingly non-specific manner. In contrast, use of the purified anti-Xrcc3 antibody produces signals referred to as nuclear foci, which are characteristic of many DNA damage signaling and repair proteins. In this case, foci are also observed in the cytoplasm and Xrcc3 also appears to aggregate near the perinuclear region. Using the example of human Xrcc3, in addition to human Rad51 and Rad51 C, in the next section a critical set of controls are described [B. T. Bennett, K. L. Knight, J. Cell. Biochem. 96 (2005) 1095-1109] and it is suggested they be included in all immunofluorescence studies to ensure proper interpretation of images.

### Assessing the Quality, Specificity and Usefulness of Antibodies for Immunofluorescence Protocols

In the DNA damage signaling and repair literature, controls are sometimes included showing antibody specificity using Western blot analyses of extracts from cells depleted for the target protein, either genetically or using RNAi methods. However, there are far fewer reports showing loss of immunofluorescent signals using these methods. In fact, the complete or near complete loss of immunofluorescence upon depletion of the target is one of the best tests of antibody specificity, as this demonstrates the lack of any significant cross-reacting material that might interfere with interpretation of the image. Data showed antibody specificity as determined by RNAi-mediated depletion of target protein. HEK293 cells were mock transfected or transfected with the indicated siRNA SMARTpools, Dharmacon. At 48 h post-transfection cells were exposed to 8 Gy IR, grown for 2 h and fixed and stained with the indicated purified monoclonal primary antibody followed by staining with a goat anti-mouse Alexa 488 secondary antibody conjugate. Antibody dilutions were 1:1000 (primary) and 1:1000 (secondary). DNA was counterstained with Vectashield containing DAPI. Panels G and H show non-transfected HEK293 cells exposed to 8 Gy IR as above but stained only using the goat anti-mouse secondary Alexa 488 secondary conjugate. Images were obtained using a Leica SP2 confocal system. Image acquisition parameters were identical for each collection. This data provided examples of this type of analysis. HEK293 cells were transfected with small interfering RNA duplexes (siRNA; SMARTpool, Dharmacon) specific for human Rad51, Rad51C or Xrcc3 (control cells were treated only with the lipid transfection reagent, Dharmafect) and grown for 48 h. At 2 h following exposure to 8 Gy IR, cells were fixed and stained with modified protein A purified antibodies against human Rad51, Rad51C or Xrcc3 followed by incubation with a highly cross-absorbed goat anti-mouse Alexa 488 secondary antibody (Molecular Probes). While control cells show characteristic nuclear foci for all three proteins, as well as cytoplasmic and perinuclear staining, cells treated with specific siRNAs showed nearly complete loss of signal (microscope settings were identical for all images). The minor amount of residual signal in some images, e.g., Rad51 and Xrcc3, is most likely due to a small background contributed by the Alexa 488 secondary antibody. RNAi-mediated knockdown of proteins was also confirmed by Western blots. Therefore, interpretations regarding protein sub-cellular distribution and alterations in this distribution in response to DNA damage can be made with much more certainty if controls such as these are used to demonstrate antibody specificity.

### Example 2

### Cell Preparation and Staining Optimization

In this section several considerations of cell growth, fixation and staining methods that are of great significance for producing optimal immunofluorescent images are discussed.

### Importance of Cover Slip Thickness in High-Performance Optical Microscopy

In preparation for immunofluorescent staining, cells are typically grown on cover slips, which can be seen as the first “lens” of a microscope objective lens. Its quality is, therefore, essential for high-quality microscopy. The most important feature of the cover slip is its thickness, since the design of a high-performance objective lens assumes a certain cover slip thickness. Most objectives have a range of cover slip thickness for which they are optimized printed on their collar. If the thickness is outside this range spherical aberrations are introduced into the imaging path, leading to potentially severe degradation of image quality. The z-resolution is especially affected by these aberrations.

Most high-quality objectives are optimized for a 0.17 mm cover slip thickness. This is especially important for objectives with high numerical apertures (NA), which provide the highest resolution, and objectives optimized for water immersion or dry usage, due to the large refractive index mismatch compared to the glass. Objectives with correction collars often allow adjustment for different cover slip thicknesses (1.2 NA water immersion Zeiss 40×, 63×, Leica 63×: 0.14-0.18 mm). Thickness (0.17 mm) is, therefore, suited for the largest variety of high-NA objectives (with or without a correction collar). The industry standard, #1.5 (equivalent to 0.16-0.19 mm), is typically the best match. However, in practice many manufacturers deliver cover slips with thicknesses at the upper edge of the specified range. For critical applications the thickness of the cover slips should, therefore, be checked with a micrometer. Alternatively, more tightly specified cover slips are available at higher prices. For this assay, one can use a 96 well or other higher well count plates that do not have the traditional molded plastic bottoms and use plates that are specially made with the correct glass and thickness, this will only increase the resolution of the image and increase the sensitivity of determining the effect of the perturbation.

### Fixation Methods

A variety of cell fixatives and permeabilization agents are available, with the most commonly used being formaldehyde/paraformaldehyde (PFA) and methanol [S. P. Wheatley, Y. L. Wang, Methods Cell Biol. 57 (1998) 313-332; R. Brock, I. H. Hamelers, T. M. Jovin, Cytometry 35 (1999) 353-362]. In the DNA damage signaling and repair literature the most frequently used fixation protocols are (i) 1-4% PFA with 0.1-0.5% Triton X-100, (ii) 1% formaldehyde on its own, (iii) a 1:1 mixture of methanol:acetone, and (iv) methanol on its own at −20° C. Similar results have been found in studies using 4% PFA with 0.1% Triton X-100 for 10 min, or −20° C. methanol on its own for 8-10 min. Both protocols suffice to fix and permeabilize cells with no apparent distortion of cellular ultrastructure and protein positioning as judged by separate stainings for actin, a- and 3-tubulin, nuclear pore and nucleoplasmic proteins, and chromatin components. Certain fluorophores are quenched to varying degrees by PFA or methanol, and control studies can be included to assess this. ˜20° C. methanol was used for the work presented here. For studies using high resolution 4Pi microscopy [J. Bewersdorf, B. T. Bennett, K. L. Knight, Proc. Natl. Acad. Sci. USA 103 (2006) 18137-18142] an important additional fixation step of 4% PFA for 5 min following staining with primary and secondary antibodies is included in order to both fix the position of the antigen-antibody complexes which prevents some time-dependent dissociation, as well as to prevent the positional fluctuation (restricted Brownian motion) of the secondary fluorophore conjugates that can actually be observed at the resolution provided by 4Pi microscopy. This technique is also useful for the invention described herein as long time periods of image collection often can be used.

### Blocking Agents

To reduce background fluorescence, sample preparation typically includes a blocking step. Most frequently the blocking buffer is PBS with one of the following agents: (i) 1-5% BSA IV; (ii) 2-5% non-fat dry milk; (iii) 5-10% goat serum plus 0.1% NP-40; or (iv) 10% fetal calf serum. When blocking with serum, the general rule is to use a normal serum from a species different from that used to generate the primary or secondary antibodies. Despite the normal precautions, varying levels of background are observed when using certain traditional blocking agents. However, it was that background can be virtually eliminated with a marine blocking agent because it has essentially no antigens that can cross-react with secondary antibodies derived from commonly used mammalian species, e.g., rabbit, mouse, donkey or goat. Background fluorescence was compared when fixed HeLa cells are blocked with a marine agent (MAXblock™, Active Motif), 5% non-fat dry milk or 5% BSA IV, followed by incubation with an anti-rabbit secondary Alexa 647 conjugate at a 1:250 dilution. This dilution is less than what is typically used, e.g., 1:1000-1:2000, to exaggerate the potential appearance of non-specific background signal. The marine blocking agent virtually eliminates any background staining whereas the non-fat dry milk and BSA show background that would interfere with interpretation of the image. Marine blocking agents are very cost effective compared to BSA IV or various sera. They are found with increasing frequency in the immuno-literature and are the product of a simple fish serum treated for sterility.

### Double Staining Methods to Assess Proximity or Colocalization

Colocalization analysis is frequently used to discern the spatial relationship of the numerous proteins and chromatin components that form visible clusters following exposure of cells to genomic insults.

Further discussion of colocalization analysis relevant to super-resolution microscopy is presented below. Here, an experimental consideration that is particularly relevant to the field of DNA damage signaling and repair is given. To briefly summarize the following description, the use of antibody mixes or cocktails containing IgGs from different species can enhance or create the appearance of colocalization if one of the epitopes is in significant excess over the other.

It has long been assumed that the use of antibody mixes or cocktails, with each antibody directed against a different target, is acceptable as long as the antibodies derive from different species, e.g., a rabbit polyclonal and a mouse monoclonal. In experiments shown here a dramatically different outcome regarding colocalization of the human Rad51 protein and γH2AX is found when using an antibody cocktail staining procedure vs. a sequential staining protocol. γH2AX is a phosphorylated version of the histone variant H2AX that in mammalian nuclei appears very rapidly following exposure of cells to DNA damage. γH2AX has been widely used as a marker for DNA double strand breaks following its discovery by the Bonner group in 1998 [E. P. Rogakou, D. R. Pilch, A. H. On, V. S. Ivanova, W. M. Bonner, J. Biol. Chem. 273 (1998) 5858-5868]. In numerous studies proteins are shown to colocalize with γH2AX and from methods descriptions it appears that mixtures of primary antibodies, and subsequently secondary antibodies, are used for immunostaining [C. Lukas et al. Nat. Cell Biol. 5 (2003)255-260; S. Bekker-Jensen et al. J. Cell Biol. 173 (2006) 195-206; T. T. Paull, E. P. Rogakou, V. Yamazaki, C. U. Kirchgessner, M. Gellert, W. M. Bonner, Curr. Biol. 10 (2000) 886-895; I. M. Ward, J. Chen, J. Biol. Chem. 276 (2001) 47759-47762; T. Taniguchi et al. Blood 100 (2002) 2414-2420; K. Nakanishi Nat. Cell Biol. 4 (2002) 913-920; M. Tarsounas, Oncogene 22 (2003) 1115-1123; M. Tarsounas Philos. Trans. R. Soc. Lond. B Biol. Sci. 359 (2004) 87-93; C. Lukas, EMBO J. 23 (2004) 2674-2683; Z. Lou Mol. Cell 21 (2006) 187-200.]

Two immunostaining protocols were compared, one using antibody cocktails and another in which antibodies are applied sequentially. Primary antibodies were a purified rabbit polyclonal against human Rad51 (Novus) and a purified mouse monoclonal against γH2AX (Upstate—now Millipore). Secondary antibodies were either highly cross-adsorbed donkey anti-rabbit Alexa 488 and donkey anti-mouse Alexa 647 conjugates, or the same with goat as the host ensuring that there is no host secondary effect. HEK293 cells were exposed to 8 Gy IR, cultured for 2 h, fixed in methanol (−20° C., 8 min) and blocked overnight using a marine blocking agent (MAXblock™, Active Motif). Using the antibody cocktail protocol, fixed cells were incubated with a mixture containing the anti-Rad51 and anti-γH2AX antibodies, each at a 1:1000 dilution, for 1 h at 37° C. Cells were washed in PBS (5 changes for 5 min each), then incubated with a cocktail containing the anti-rabbit and anti-mouse secondary antibodies, each at a 1:1000 dilution, for 1 hat 37° C. and washed again. Coverslips were mounted using Vectashield with or without DAPI, sealed in polyurethane and stored at 4° C. in the dark. Using the sequential staining protocol, fixed cells were stained first with the rabbit anti-Rad51 antibody followed by the anti-rabbit secondary, then the anti-γH2AX antibody followed by the anti-mouse secondary. All antibody dilutions, washes and coverslip mounting were done as above. Cells were visualized using a Leica TCS SP2 AOBS confocal microscope and image processing performed using the accompanying Leica software. Microscope settings were identical for all images taken in both staining protocols.

Using antibody cocktails, Rad51 and γH2AX appear exclusively in the nuclei. The merge image and the accompanying cytofluorogram show nearly perfect coincidence of the fluorescent signals. Using the sequential staining protocol, it was found that while γH2AX appears exclusively in the nuclei as expected, Rad51 shows both distinct nuclear focal staining, as well as a robust cytoplasmic signal. Additionally, the merge image and accompanying cytofluorogram show little coincidence of the nuclear Rad51 and γH2AX signals. These same differences between the cocktail vs. sequential protocols were observed using different primary and secondary antibodies, in this case a purified mouse monoclonal against Rad51 and a purified rabbit polyclonal against γH2AX.

It is also worth noting that the 488 and 647 nm fluorophores have no spectral overlap, which eliminates the possibility of bleed-through. Additionally, these were sequential scans requiring that only one laser line be active at a time, which again eliminates any possible bleed from channel to channel.

There are many accounts in the literature showing significant amounts of cytoplasmic Rad51 in various mammalian cell lines [K. Yoshikawa et al. Int. J. Cancer 88 (2000) 28-36; A. A. Davies Mol. Cell 7 (2001)273-282; J. Essers EMBO J. 21 (2002) 2030-2037; M. Kraakman-van der Zwet et al. Mol. Cell. Biol. 22 (2002) 669-679; N. Liu, C. S. Lim, J. Cell. Biochem. 95 (2005) 942-954; E. Honrado J. Clin. Oncol. 23 (2005) 7503-7511; E. Mladenov, B. Anachkova, I. Tsaneva, Genes Cells 11 (2006) 513-524; S. E. Henson Mutat. Res. 601 (2006)113-124], and in some cases cytoplasmic Rad51 is clearly apparent by immunofluorescence [K. Yoshikawa et al. Int. J. Cancer 88 (2000) 28-36; J. Essers et al. EMBO J. 21 (2002) 2030-2037; M. Kraakman-van der Zwet et al. Mol. Cell. Biol. 22 (2002) 669-679.] Further, it was shown that the fluorescence signal for Rad51, as well as Rad51C and Xrcc3, is eliminated when cells are depleted of the protein using specific siRNAs, thus strongly supporting the veracity of the observed cellular localization.

Regarding the significant difference in fluorescent signal appearance using the cocktail vs. sequential staining protocols, several experiments were performed to ask whether the staining of γH2AX somehow influences that for Rad51, reasoning that the possible extreme excess of γH2AX antigen following DNA damage relative to Rad51 may negatively influence Rad51 staining. Using primary antibodies against γH2AX and Rad51 that were both rabbit polyclonals, a sequential staining procedure was performed in which cells were incubated first with the anti-Rad51 polyclonal, washed extensively, incubated with the anti-γH2AX polyclonal, washed extensively again and incubated with an anti-rabbit Alexa 488 secondary. It was expected that an image similar to the merge indicative of nuclear localization of γH2AX would be seen, and both nuclear and cytoplasmic localization of Rad51. It was found that only nuclear staining occurred. Identical images were seen using mouse monoclonals against both proteins followed by incubation with an anti-mouse Alexa 647 secondary. Therefore, given the opportunity to interact with both primary antibodies, the secondary appears to see only the anti-γH2AX primary antibody. These results suggest that the primary γH2AX antibody out-competes the primary Rad51 antibody for interaction with the secondary antibody. This is most likely explained by the excess epitope availability of γH2AX relative to Rad51 following DNA damage. Therefore, it stands to reason that the appropriate Rad51 signal can be recovered by reducing the amount of γH2AX primary antibody used in the staining procedure. The images showed the results of such an experiment. In this case cells were stained using our standard sequential staining procedure and a 1:1000 dilution of anti-Rad51 rabbit primary antibody, but the anti-γH2AX rabbit primary antibody was used at a 1:10,000 dilution. Subsequent staining with the anti-rabbit Alex 488 secondary reveals fluorescent signal in both the nucleus and cytoplasm. This result suggests that the amount of γH2AX epitope available following the response of cells to DNA damage creates a situation where there is such an excess of primary γH2AX antibody that it out-competes the Rad51 primary for interaction with the secondary antibody.

These data strongly support the idea that relative epitope abundance can contribute to the misleading appearance of colocalization if antibody cocktails are used, particularly for experiments involving high abundance epitopes such as γH2AX following DNA damage. Interestingly, if the use of antibody cocktails had shown excessive cytoplasmic staining, a methodological red flag would have been raised. Unfortunately, the exclusive nuclear staining of this pair is easily rationalized in terms of DNA damage and repair events, and this particular procedural caveat went unnoticed. If antibody cocktails are used, it is very important to provide controls demonstrating that each antibody behaves similarly when used either individually or in a cocktail. While it may seem that the multiple steps used in the sequential staining protocol are excessively time consuming, use of this procedure avoids the possible misleading appearance of inappropriate colocalization or the enhancement of an otherwise limiting amount of signal coincidence. For the experiments described herein the use of this testing proves not only to support proper immunofluorescence in super-resolution microscopy but in high-content screening as described herein.

### Example 3

### Super-Resolution Microscopy Exemplified by 4Pi

Image resolution is determined by (i) the pixel size, which can be easily controlled by choosing a suitable magnification of the microscope and (ii) optical resolution. Choosing a pixel size approximating half the optical resolution value renders it non-limiting and is, therefore, highly recommended. The optical resolution, however, is fundamentally limited by diffraction and cannot be adapted easily. Even the best objectives with the highest NAs available provide only about 200 nm resolution in the focal plane. The depth (or axial) resolution is at least 2.5 times worse, making it impossible to differentiate between two objects that are axially separated by 500 nm or less. Using a regular wide-field microscope, axial discrimination, especially of bulky objects within a DAPI-stained nucleus, becomes extremely difficult or even impossible due to the strong out-of-focus background contribution to the signal. In this case, colocalization studies are often reduced to analyzing two-dimensional (2D) images generated from single recordings or projections of three-dimensional (3D) stacks along the axial direction. This reduces the 3D nuclear architecture to a 2D view and, therefore, weakens statements based on resulting colocalization studies. Especially for objects present at high density it is difficult to decide whether two objects really coincide in all three dimensions or are located at different depths of the sample. Confocal laser scanning microscopy provides true 3D resolution by suppressing out-of-focus light. For colocalization studies of inherently 3D objects, confocal microscopy or alternative true 3D-imaging techniques are, therefore, recommended.

To observe fine structures at the 100 nm level or to narrow colocalization information down to these values or less, super-resolution microscopy techniques are necessary. Recent years have seen the emergence of several alternative techniques such as STED, RESOLFT, FPALM, PALM, STORM and others summarized by Stefan Hell in a recent review [S. W. Hell, Science 316 (2007) 1153-1158]. The focus here is on 4Pi micros-copy, the first one of these techniques to become commercially available and provide 3D imaging capabilities suitable for the analysis of nuclear structures.

4Pi microscopy is a laser scanning microscopy technique that enhances the axial resolution of standard confocal microscopy 5-7-fold. This is achieved by doubling the solid aperture angle used for excitation and/or detection using two opposing objectives that focus into the same spot in an optically coherent arrangement. In the favored Type C variant of 4Pi microscopy the excitation wavefronts passing through the two objectives interfere constructively, and the focus center-emitted light is co-lected by both objectives and recombined constructively. This results in a sharp central focus of 100 nm axial width [H. Gugel et al. Biophys. J. 87 (2004) 4146-4152]. Side maxima, which additionally appear, are removed by processing the recorded data with an image restoration (deconvolution) algorithm. Imaging the cell nucleus with 4Pi microscopy, as well as with most other super-resolution microscopy techniques, requires special care due to the strong refractive index mismatch between the nucleus and typical aqueous embedding media such as PBS. Matching the refractive index of the embedding medium with the nucleus by using glycerol at a high concentration (80-90%) and using glycerol immersion lenses (100×/1.35 NA), or alternatively 2,20-thiodiethanol and oil immersion lenses (100×/1.46 NA) [T. Staudt et al. Microsc. Res. Tech. 70 (2007) 1-9] provides conditions suitable for super-resolution imaging of the nucleus.

A single optical section taken from a 3D data set of a HeLa cell nucleus stained for histone H2AX, comparing standard confocal microscopy to 4Pi microscopy was shown. The clusters of H2AX observed are much more clearly resolved in the 4Pi data set and allow for automatic differentiation and quantification [J. Bewersdorf, B. T. Bennett, K. L. Knight, Proc. Natl. Acad. Sci. USA 103 (2006) 18137-18142]. The superior resolution offered by 4Pi allowed, for example, the determination that the diameters of H2AX clusters are 100-600 nm less than the much larger diameters of γH2AX clusters seen following exposure of cells to IR [J. Bewersdorf, B. T. Bennett, K. L. Knight, Proc. Natl. Acad. Sci. USA 103 (2006) 18137-18142].

Colocalization can be quantified best by analysis using multidimensional histograms, or “cytofluorograms”, where each axis rep-resents one color channel. Every pixel (or voxel) of a 2D (or 3D) data set is added to this histogram according to its values in each of the analyzed color channels. High levels of colocalization lead to an accumulation of pixels along the diagonal of the histogram while non-colocalized staining shows up as signal along the axes. A problem often occurring in fluorescence microscopy is the large fraction of dark background pixels. They are represented close to the origin of the histogram as a very large peak with an amplitude that can easily exceed the values representing brighter structures by a factor of 1000 or more. To better visualize the more interesting fraction of pixels representing brighter cellular structures, a logarithmic color table can be used for the amplitudes in the histogram or cutting off the histogram amplitudes at lower levels. Colocalization can be quantified by comparing the integrated amplitudes in different regions of interest (ROIs) in the histogram. Care must be exercised in defining the ROIs so as not to include the large amplitudes of the background peak close to the origin. This method is preferred over conclusions from purely visual interpretation of the recorded images. Judging by eye often underestimates the contribution of dimmer structures in the sample.

Colocalization studies depend critically on the achieved resolution in the analyzed image data. 4Pi microscopy, for example, enabled the distinguishing of neighboring clusters of H2AX and γH2AX at 100 nm resolution. This was seen where signals of neighboring clusters are spatially separated and a cytofluorogram of the 4Pi 3D data sets reveals that, in fact, H2AX and γH2AX clusters do not colocalize [J. Bewersdorf, B. T. Bennett, K. L. Knight, Proc. Natl. Acad. Sci. USA 103 (2006) 18137-18142].

While 4Pi microscopy provides a 5-7-fold improvement in 3D resolution over standard confocal microscopy, the achieved 100 nm resolution is still more than a factor of 10 larger than the diameter of a typical protein. Some of the more recent super-resolution microscopy techniques, namely STED and the FPALM/PALM/STORM techniques, have reached 20-30 nm resolution over the last years [S. W. Hell, Science 316 (2007) 1153-1158]. In the DNA damage and repair field, the 3D imaging variants of these instruments are of particular interest because they provide the means to explore complex structures in the nucleus at nearly molecular resolution. 2008 saw the emergence of 3D-STORM, 3D-FPALM and isoSTED [B. Huang et al. Science 319 (2008) 810-813; M. F. Juette et al. Nat. Methods 5 (2008) 527-529; R. Schmidt et al. Nat. Methods 5 (2008) 539-544], which provide 20-75 nm resolution in all three directions simultaneously. Multi-color and live cell imaging is also being developed at a fast pace [S. T. Hess et al. Proc. Natl. Acad. Sci. USA 104 (2007)17370-17375; H. Shroff et al. Proc. Natl. Acad. Sci. USA 104 (2007) 20308-20313; H. Shroff et al. Nat. Methods 5 (2008) 417-423; M. Bates Science 317 (2007)1749-1753; M. Bates, B. Huang, X. Zhuang, Curr. Opin. Chem. Biol. 12 (2008) 505-514; J. Folling et al. Nat. Methods 5 (2008) 943-945]. The resolution of these new microscopes a-proaches the size of the fluorescent labels used, now posing a new limitation, which only a few years ago was still far out of reach. While this problem is being tackled, however, the time is ripe to apply these techniques already at the “moderate” resolution of 20-75 nm to studies of DNA damage signaling and repair, thus providing new visual insights into these complex processes at resolving volumes approximately 1000-fold smaller than achieved by conventional confocal microscopes.

### Example 4

### Mathematics

Many cells (typically >1,000) over a very large field of view were imaged. At least two stainings were used simultaneously. An optional third staining, here propidium iodide (PI), was also used as a nuclear locator.

### Example:

Staining 1: p-ATM

Staining 2: gamma-H2AX

The cells were imaged in 3 channels which corresponded to the three stainings. The third channel was used for cell profiling.
In a first data processing step, cells were automatically identified in the images by their signal and/or shape in one or both of the recorded channels. Alternatively, cells were identified using a nuclear counterstain and then the activator(s) were read. This resulted in a total number of cells from counting the identified cells: N The signal of two channels was determined for every cell c (c denoting the number of the cell ranging from N to N+) and stored in S1(c) and S2(c) (the subscript number denotes the channel number).
From the distributions S1(c) and S2(c), characteristic numbers were derived. For example (at the example of channel 1, same valid for channel 2).

Average signal of all cells: <S1>

Number of “positive” cells (i.e. affected cells with S1(c)>threshold 1): N1+

Fraction of positive cells: F1=N1+/N

Average signal of positive cells: <S1+>

The imaging and analysis procedure were repeated for cells which were fixed at different time points t and under influence of different drug doses dd. For every condition (time point and drug dose), the parameters above were determined. This resulted in two-dimensional data sets

N(t, dd)

<S1>(t, dd), <S2>(t, dd)

N1+(t, dd), N2+(t, dd)

F1(t, dd), F2(t, dd)

<S1+>(t, dd), <S2+>(t, dd)

Depending on the application and specific question, specific values were extracted from these data sets.

The values above plotted versus t feature characteristic shapes in their time dependence, for example they decay exponentially. Customized to a particular application, a specific value was extracted at a particular time point, e.g. the number of remaining cells 2 hours after drug treatment: N1+(t0=2 hrs, dd) (t0=2 hrs). Plotting these values now versus dd, results in a single curve. From this curve, e.g., the specific drug dose dd0 was extracted at which more than 99% of the cells are positive.

This procedure therefore allowed to extract a single value dd0 was be extracted from the wealth of data. Multiplied by a scaling factor which translates this value to a specific dose required for the treatment of the patient (the donor of the used cells), this procedure represented a quantitative process to determine the best personalized treatment.

The procedure was also repeated for different drugs and different fluorescent markers to get a more complete picture.

### Example 5

Hela, MCF7, as well as primary cells from a patient were cultured for growth in this assay. Cells were obtained and seeded initially at ˜100 cells per well/96 well plate . The cells were grown in DMEM, with 10% Fetal Falf Serum, ultra pure, along with 5% Pen/Strep. The cells after being plated were placed in an incubator at 37° F. with 5% CO2 and grown for a period of 28 hours to allow for adhesion and division . If the cell took to the plate and were growing normally they were allowed to continue for an additional 24 hours. Cells were then treated with either Taxol, Doxirubicin, or Ionizing Radiation or a combination of all 3. All drugs were added in the logarithmic titrations shown in the data at time point zero. The cells were then collected (fixed with −20 degree methanol for 7 minutes, washed with PBS, washed again and stored in PBS until the entire plate of titrations were finished. After collection, the nucleus of each cell was stained using PI (far RED) by adding PI for 15 minutes and then washing 3× the cells with sterile PBS . Once the nuclei had been stained, each well was stained for either phosphor ATM or gamma H2AX or both using primary antibodies at a dilution of 1:1000 in a PBS/Block mix for 1 hour at 37 degrees. The cell were then washed 3× with PBS and then treated with the appropriate secondary . Here, the experiment first staining for only one marker using a green label so that the read was unpolluted by any other color that could show cross reactivity with the green (as there is RED, PI staining the nucleus already, which has no cross reactivity), once the data was collected and the data satisfied that the independent signal was reproducible, a double staining was performed by adding the second marker and using a orange dye with a wavelength of 555. When the data was collected the results for each of the markers was unpurturbed by the addition of the second marker in the same staining.

### Results:

Raw images of fixed cells with the nuclear counter-stain (PI—far red emission) were taken and the cells were profiled according to what was considered morphologically acceptable for this individual study. For this experiment, size of the nuclei was used.

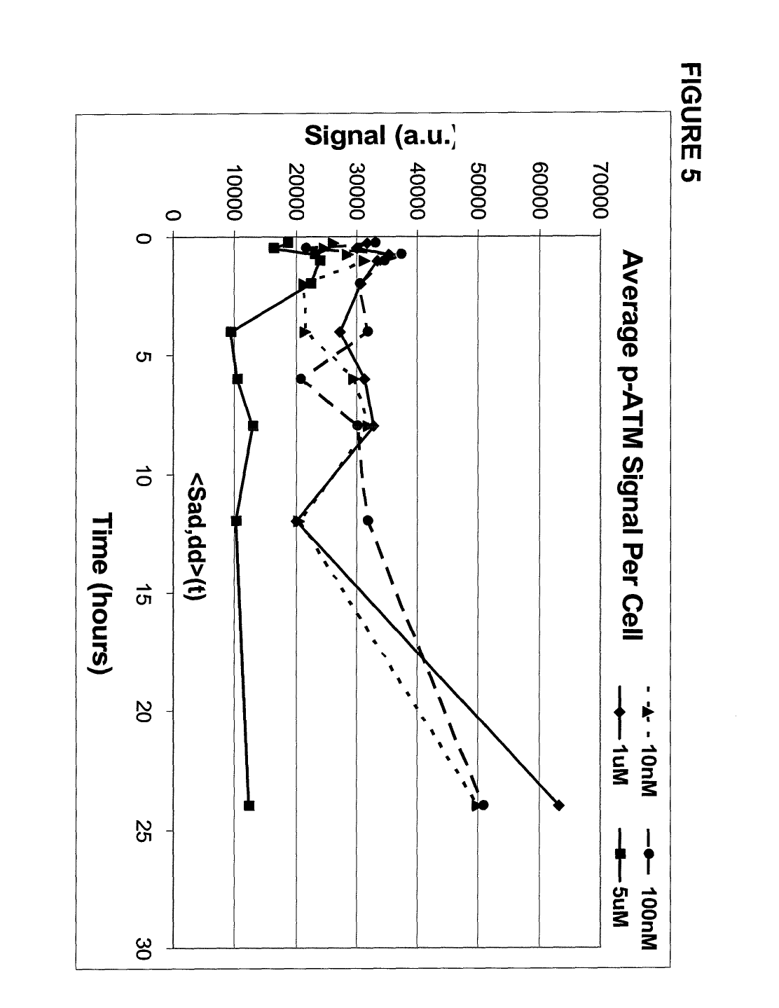

FIG. 4 shows the titration of Taxol, a common cancer drug on estrogen positive, immortalized breast cancer cell line, MCF7. The doses given were given for in the amounts shown, 10 nm, 100 nm, 1 um and 5 um. The drug was given in a 1 hour pulse and time zero, or the point at which the information is collected begins immediately following that pulse. For this experiment, the interes was in the fraction of cells in the identified population of “normal” nuclei and how they respond to the 4 titrations. First, the maximum response, which in this case is an approximate 100% induction rate of γH2AX was achieved seemingly with all 4 doses even though the range between the lowest dose at 10 nm and the highest at 5 um is large indeed, but as shown in the box at the 4 hour post treatment time point all (plus or minus error, bars not shown) appeared to reach 100% effected. Second, if one makes no assumptions at this point because the population death is not represented here, one would believe that the 5 um was most effective as the fluorescence drops of significantly, reaching its lowest point at hour 12 and that +/− error after 12 hours the 5 um dose was relatively stagnant in that there is little change out to ˜25 hours post treatment. Next, the lowest dose (10 nm was the next most effective. The 10 nm dose droped off most significantly when compared to the highest dose.

In addition, the middle doses of 100 nm and 1 um demonstrated data as well. Here the 1 um treatment shows again that drop off in fluorescence, but in a much smaller portion of the population and like the others at 12 hours.

This data shows that treatment with Taxol, one of the most commonly prescribed drugs for breast cancer, was in fact at the highest dose only 30% effective on a population.

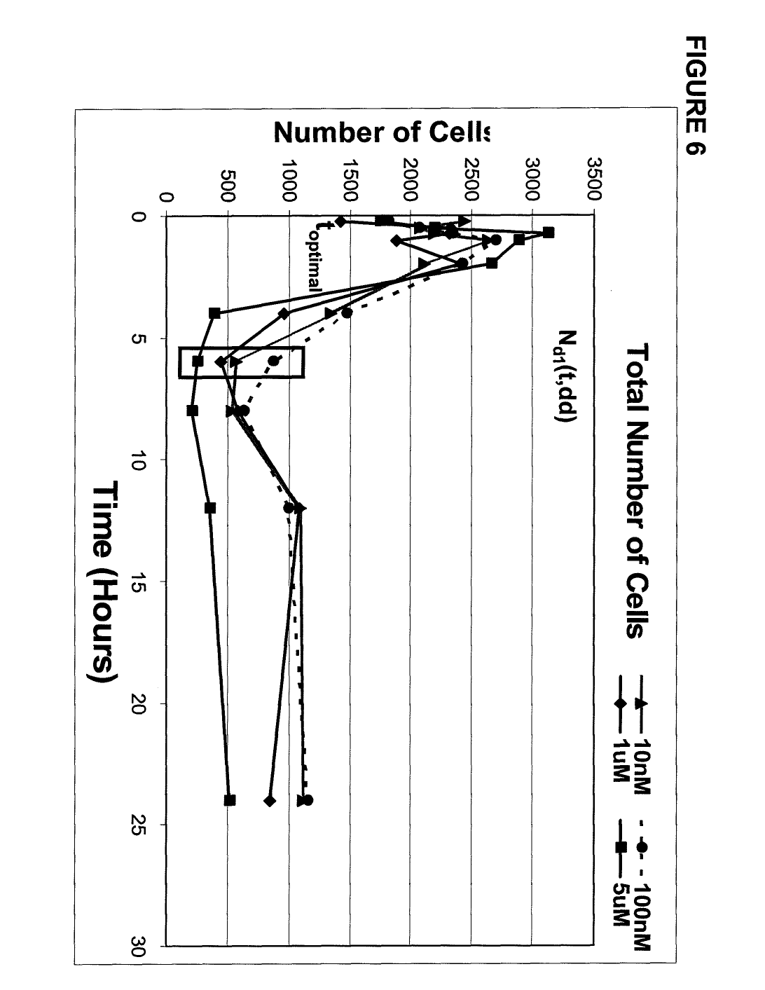

The same cell population was also examined, but this time for p-ATM. FIG. 5 shows that the 5 um dose signal drops from a maximum arbitrary fluorescent signal unit (a.u.) from an initial elevation to ˜2300a.u. to ˜1000a.u. which shows that the activity of the kinase that signals the damage or perturbation to the cell drops within 4 hours (again noting that there was a maximum γH2AX activity at 4 hours for all doses) to its lowest point of 1000 a.u. Considering standard error there appears to be no activity from the cell population for the 5 um dose following 4 hours. Furthermore, the other 3 doses all showed increased p-ATM activity over the 5 um dose and all at the 12 hour time point rapidly increased in their signal or activity as it pertains to the signal, noting that in the γH2AX data the 12 hour time point also showed increased signal, this taken together, knowing that p-ATM is the activator for gamma H2AX, demonstrating that the two markers are correlative and interact in a pathway. The data also shows that the 3 lowest doses caused a rapid increase in activity at 12 hours—while the 5 um dose showed very little

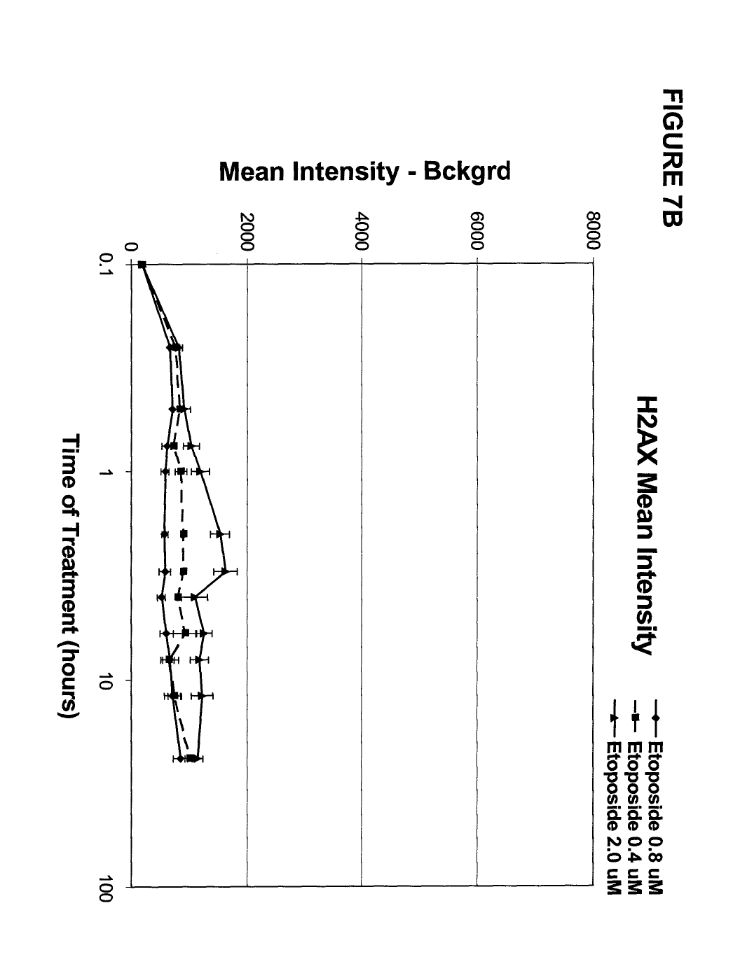

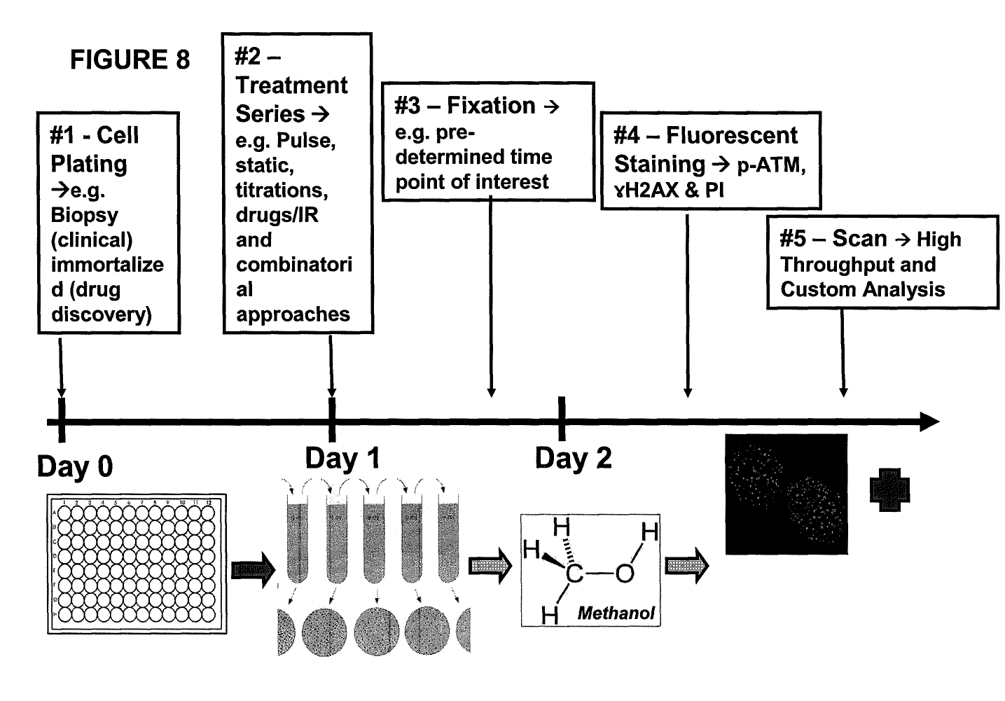

To show that the sensitivity of this assay, etoposide was used to treat a population of benign HeLa cells. Etoposide induces double strand DNA breaks by stalling replication forks and so the breaks are a secondary effect of the etoposide and therefore further lends to our sensitivity. FIG. 7A show that when dosed at 0.08 um, 0.4 um and 2 mm, both the activator (p-ATM) and the marker (gamma H2AX) can be detected for the sensitivity of the dose. In fact, the levels of p-ATM were plotted with distances between them equal to that of the doses.

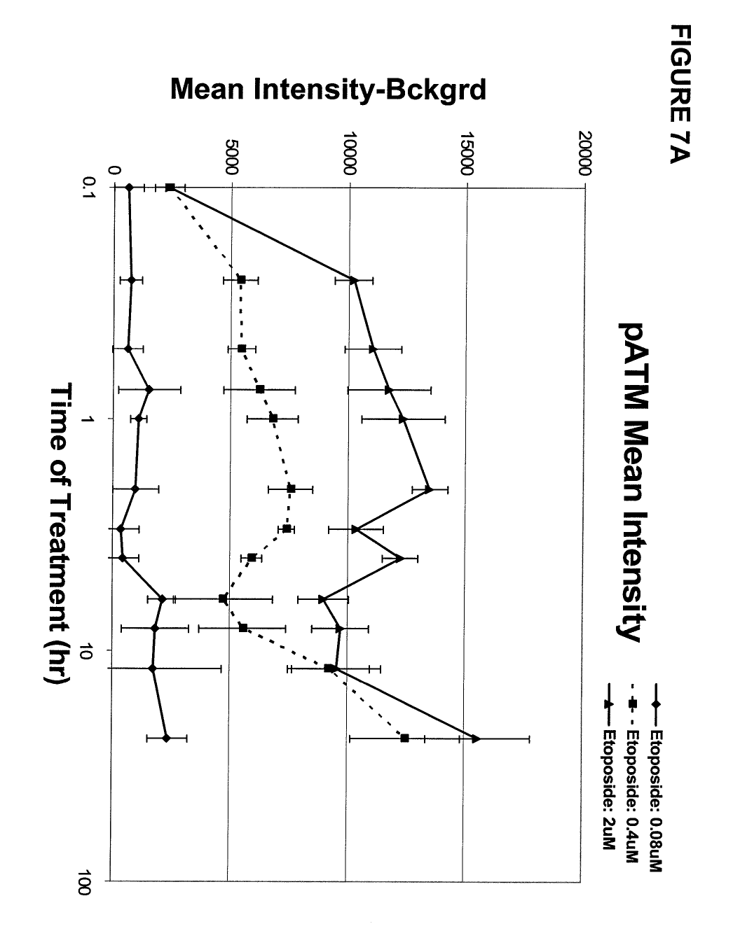

N was then investigated, and the data revealed that all treatments were effective in killing cells (See FIG. 6). Even the lower doses, all dd killed cells, and all were most effective at 4 hours, a time point consistant with the data shown in FIGS. 5 and 7A, where both p-ATM and γH2AX demonstrated a level of activity. Furthermore, the data revealed that the kill is in fact dose dependent noting that the 5 um is the most effective and the lower 100 nm dose the least effective at the datas lowest point 4 hours, meaning the assay is sensitive in both the nm and um range. In addition, after 4 hours the activity of the cells, or the cell numbers, through division begin to increase. Also, the data revealed that the 5 um dose, does not kill the entire population as also shown in the γH2AX Figure.

### Example 6

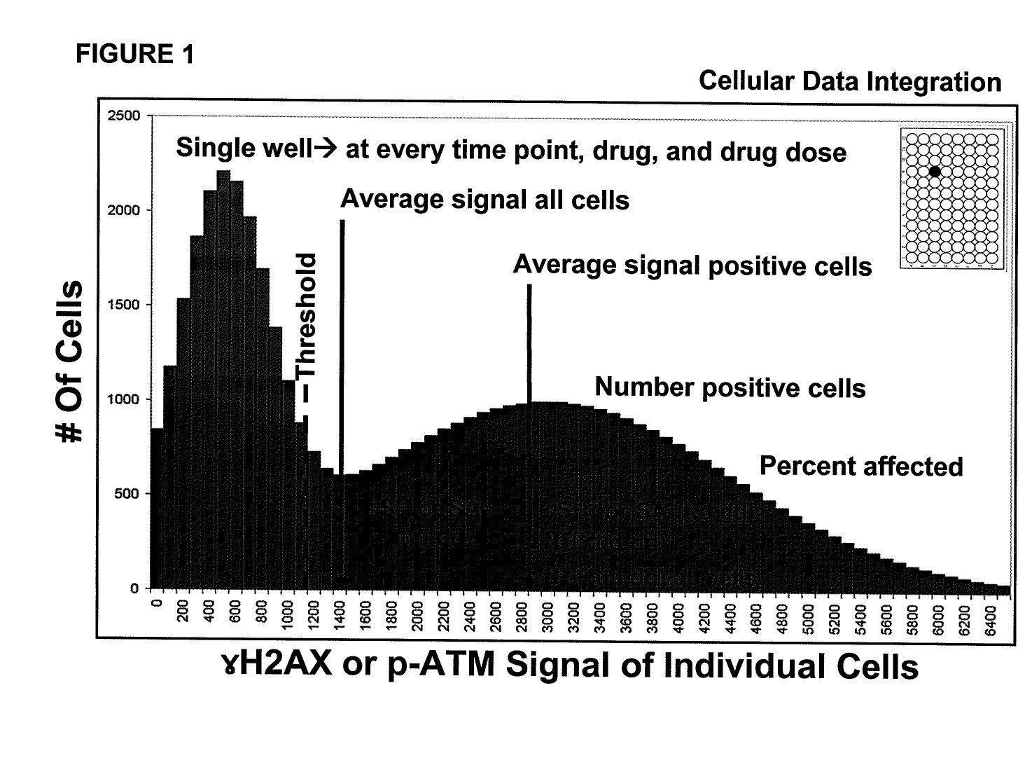

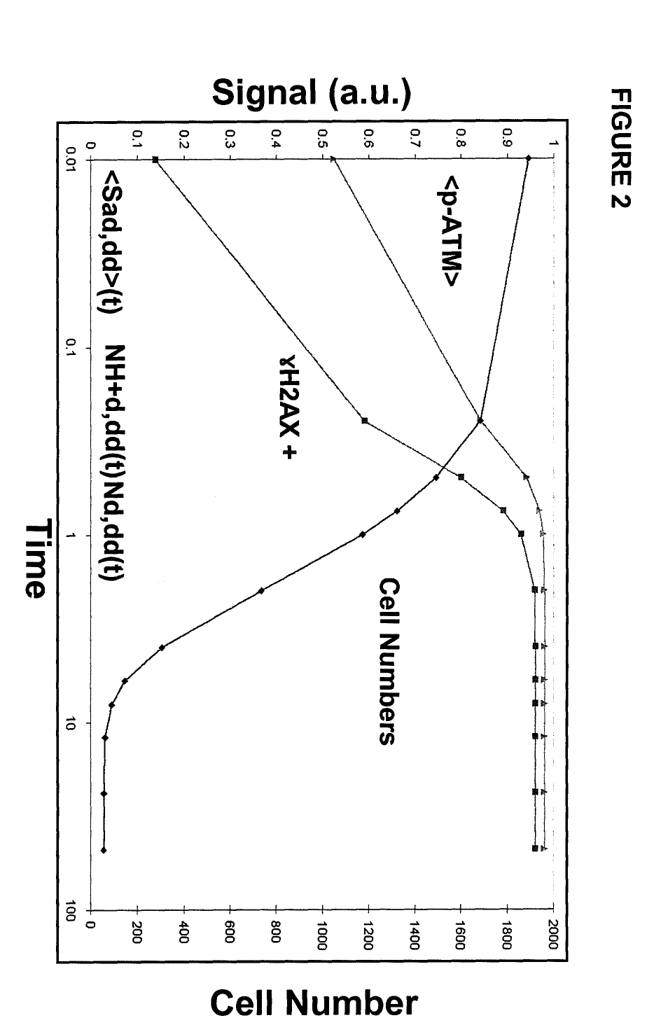

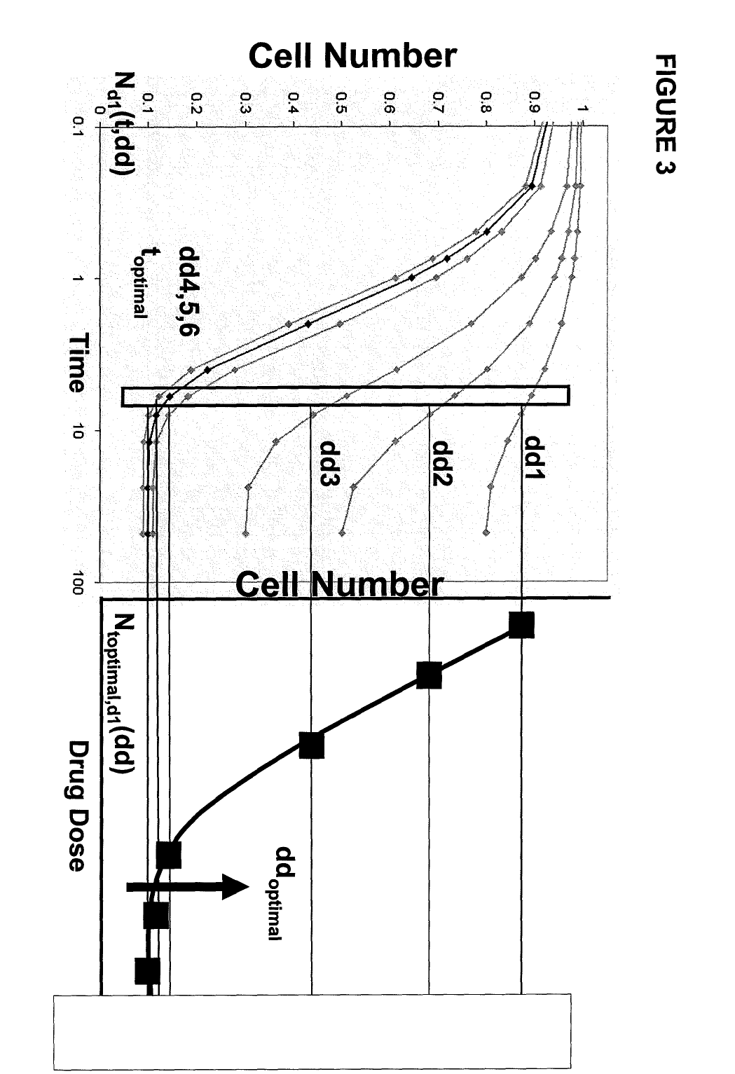

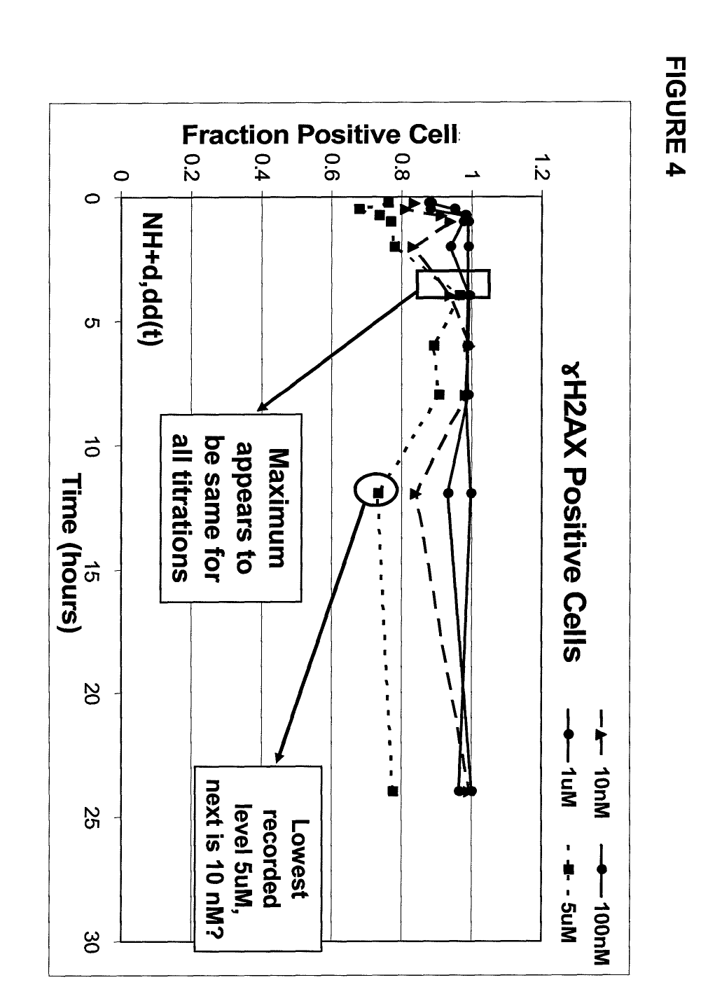

FIG. 1 shows a gaussian fit of what would be considered a typical curve for a healthy population undergoing mild stress to the cellular environment. The Y axis is the # of cells and the X axis is an arbitrary unit of measurement for fluorescent signal. Even cells without labeling demonstrated some level of fluorescence and therefore the background has to be accounted for, if one was to measure the cell population with any laser line at high intensity there would be a certain amount of auto fluorescence. In FIG. 1, the highest cell count number has the lowest signal. This is considered background and is therefore eliminated in an active computer driven applied threshold—after the threshold is the data moving left to right. The first thing encountered was the average signal for all cells or the mean signal as the curve increases and reaches it highest point or the point were the signal is the most intense and was measured and the totality of the average signal for positive cells as fluorescence increased with perturbation. In FIG. 1, this population has a range of signal from intense to weak—(˜1K cells showed an ˜3100 fluorescent signal); however, not all cells showed this and this signal increased were we see that ½ the number of cells 500 have a signal of ˜4400 and at 250 cells the signal again goes up, showing that a small percentage of the cells have the strongest signal—this means that if the final goal was apoptosis it may only be accomplishing in small numbers. This became even more evident when the background was removed and only positive cells were were viewed and the total number of cells exceeded 2000 but positive was ½.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of evaluating an effect of an agent on a cell population, comprising:
a) profiling cells in a cell population;
b) contacting at least the profiled cells with an active agent;
c) contacting at least the profiled cells with at least one marker agent;
d) measuring the response of the active agent on the profiled cells.

2. The method of claim 1, wherein a criteria of profiled cells comprises expression level of extracellular or intracellular markers, nuclear antigens, enzymatic activity, protein expression and localization, cell cycle analysis, chromosomal analysis, cell volume, and morphological characteristics like granularity and size of nucleus, size and redistribution of nucleoli, membrane change, or the number of nuclear pores.

3. The method of claim 1, wherein a criteria of profiled cells comprises the expression of surface markers of CD45, CD34, CD33, CD11B, or CD14.

4. The method of claim 1, wherein step b) further comprises contacting the population of cells with a predetermined concentration(s) of a second agent.

5. A method of determining an optimal dosage of an agent required to achieve a selected cellular response in a population of profiled cells, comprising:
a) profiling cells in a cell population;
b) contacting at least the profiled cells with a predetermined dose of an active agent;
c) contacting at least the profiled cells with at least one marker agent;
d) correlating marker levels with cellular viability in the profiled cells;
d) comparing the correlation data of e) with data from other dosages; and
g) determining the dosage required to achieve the selected cellular response in a population of profiled cells.

6. A method for determining cellular responses in a profiled cell population, said method comprising:
a) profiling cells in a cell population;
b) contacting at least the profiled cells with at least one first marker agent and one second marker agent, wherein the first marker agent is a cell specific marker agent and the second marker agent is different from the first marker agent;
c) contacting at least the profiled cells with an active agent;
d) determining the amount of the second marker agent in the profiled cells; and
e) comparing the level of the second marker agent in the profiled cells to the level of second marker agent in step b), wherein a change in the amount of second marker agent is indicative of a cellular response in the profiled cells.

7. An automated method for identifying cellular responses in a profiled cell population, said method comprising:
i) providing a computer, profiled cells, and a detection device configured to monitor said profiled cells and obtain cellular image data;
ii) obtaining a first set of cellular image data from said profiled cells with said detection device;
iii) automatically communicating said first set of cellular image data to said computer;
iv) defining said first set of cellular image data with said computer;
v) automatically contacting said profiled cells with a test composition to evoke a cellular response;
vi) obtaining a responsive set of cellular image data from said profiled cells upon evocation of said cellular response with said detection device;
vii) automatically communicating said responsive set of cellular image data to said computer;
viii) defining said responsive set of cellular image data with said computer;
ix) automatically identifying a first set of differences between
a) said defined first set of cellular image data and
b) said defined responsive set of cellular image data;
x) generating a signal from said computer in response to said identified first set of differences;
xi) independently automatically actuating in response to said signal one or more stimulating devices configured to interact with said profiled cells; and
xii) obtaining a second responsive set of cellular image data from said profiled cells following said independent automatic actuating of said one or more stimulating devices.
